(19) United States
(12) Patent Application Publication
(10) Pub. No.: US 2025/0082724 A1
(43) Pub. Date: Mar. 13, 2025

(54) VERSATILE IMMUNOGENIC MODULES AND NANOPARTICLES FOR TICK VACCINES

(71) Applicants: Institut Pasteur de Montevideo, Montevideo (UY); Universidad de la República, Montevideo (UY)

(72) Inventors: Matias Machado, Montevideo (UY); Agustin Correa, Montevideo (UY); Janaina Joselin Lasalvia, Montevideo (UY); Maria Soledad Valledor, Montevideo (UY); Oscar Correa, Montevideo (UY); Laura Decia, Montevideo (UY); Uruguaysito Benavides, Montevideo (UY); Alejandra Navratil, Montevideo (UY); Nahuel Santana, Montevideo (UY)

(73) Assignees: Institut Pasteur de Montevideo, Montevideo (UY); Universidad de la República, Montevideo (UY)

(21) Appl. No.: 18/883,051

(22) Filed: Sep. 12, 2024

Related U.S. Application Data
(60) Provisional application No. 63/537,861, filed on Sep. 12, 2023.

(51) Int. Cl. A61K 38/17; A61P 37/04; C07K 14/435

(52) U.S. Cl. CPC A61K 38/1767; A61P 37/04; C07K 14/43527

(57) ABSTRACT

The invention relates to self-assembling multimeric recombinant proteins composed of a plurality of polypeptide subunits, and a plurality of immunogens derived from the Bm86 protein of Tick, and their applications for preventing, and controlling Tick infestations in a subject. The invention also relates to methods for engineering and producing the self-assembling multimeric recombinant proteins.

This application claims priority benefit from U.S. provisional patent application No. 63/537,861, filed on Sep. 12, 2023, the entire content of which is incorporated herein by reference.

The instant application contains a Sequence Listing which has been submitted herewith and is hereby incorporated by reference in its entirety. Said .xml copy, created on Sep. 11, 2024, is named 150126.596328, and is 20,744 bytes in size.

### BACKGROUND

### Field of the Invention

The invention relates to compositions and methods for conferring immunity and preventing, controlling, or reducing tick infestations and the transmission of tick-borne pathogens. The invention relates to self-assembling multimeric recombinant proteins, immunogenic compositions, vaccines, methods for engineering and producing such self-assembling multimeric recombinant proteins and their applications for immunotherapy.

### Discussion of the Related Art

Ticks are hematophagous arthropods and are found worldwide, especially in tropical and subtropical countries. As ectoparasites, ticks are vectors of bacteria and viruses causing diseases to humans and animals and are responsible for inducing allergic and immunologic reactions. Ticks are known to infest a wide range of hosts from reptiles, birds to mammals including pets and cattle.

In livestock, ticks may transmit pathogens such as Babesia bovis, Babesia bigemina, and Anaplasma marginale, etiological agents of the bovine parasite sadness. In addition to tick-borne diseases, tick infestation causes weight loss, udder health problems, and milk production issues in cattle, leading to important economic losses. Rhipicephalus microplus, commonly known as the cattle tick, is the most prevalent tick affecting livestock. Current strategies aimed at combating R. microplus include herd management and chemicals usage such as acaricides and macrocyclic lactones. Unfortunately, the populations of ticks have developed resistance to such compounds due to their ubiquitous use in tick control. Moreover, chemicals cause potential hazards to the environment, meat, and milk when incorrectly applied.

Accordingly, vaccination provides an appealing alternative to chemical acaricides for their safety and cost-effectiveness. Since the first vaccination trials using crude tick extracts in 1939, several protein antigens derived from ticks have been identified as potential vaccine candidates. The list of antigens includes pro-cathepsin, vitelin, degrading cysteine endopeptidase, glycoprotein 80, larvae trypsin inhibitors, 5′-nucleotidase, ferritin 2, ubiquitin, elongation factor 1 alpha, major surface protein 1a (MSP1a), glutathione S-transferases, flagelliform silk protein, tick receptor for outer surface protein A, reprolysin, metaloprotease 4, aquaporin 1 (RmAQP1), ribosomal protein P0 (pP0), salivary gland proteins such as subolesin, akirin, 05 (Bm05), 39 (Bm39), 76 (Bm76), 91 (Bm91), 180 (Rm180), 239 (Rm239), and gut proteins: 86 (Bm86), 95 (Bm95), ATAQ. However, shortcomings are associated with most of the prior art recombinant vaccines including, poor efficacy, high genetic variability among tick populations, high cost of manufacture, and formation of inclusion bodies which can impede the production of soluble and functional recombinant proteins essential for vaccine efficacy.

There is an unmet need to design novel immunogens for vaccines that induce a potent and long-lasting immune protection against tick infestations. There is also a need for improved vaccines that can be produced easily, are efficacious and safe, and can induce long-term protection against a wide spectrum of tick species.

### SUMMARY OF THE INVENTION

The invention relates to immunogens, immunogenic nanoparticles, immunogenic compositions, vaccines, and methods to control, prevent and treat tick infestations in animals, including domestic mammals, livestock or birds. The invention also relates to improved method for producing immunogens and tick vaccines. Such immunogens comprise recombinant Bm86-derived modules (e.g., Bm86Dock or variants thereof) which displays epitopes capable of inducing a protective immune response, including the production of neutralizing antibodies. Advantageously, the Bm86Dock immunogens or variants thereof are displayed on self-assembling multimeric protein scaffolds forming immunogenic Bm86Dock-nanoparticles (Bm86Dock-NP). When administered to mammals or birds, the Bm86Dock immunogens or variants thereof confer potent immunity and long-lasting protection against ticks. Vaccine Formulations comprising Bm86Dock immunogens or variants thereof may be further formulated with adjuvants such as aluminum oxyhydroxide (AlOH).

Further aspects of the present disclosure are provided by the subject matter of the following embodiments.

Provided herein is an isolated recombinant Bm86Dock module derived from the protein Bm86 which comprises an amino acid sequence selected from the group consisting of SEQ ID NO: 2, SEQ ID NO: 3 and variants thereof, or an amino acid sequence having an immunogenic fragment consisting of SEQ ID NO: 1. The recombinant Bm86Dock module elicits an immune response.

The isolated recombinant Bm86Dock module of the preceding embodiment, comprises an immunogenic fragment consisting of SEQ ID NO: 1.

Also provided herein is a Bm86Dock immunogen for treating, controlling or preventing a tick infestation in a subject which includes: a recombinant Bm86Dock module of a protein Bm86 including an amino acid sequence selected from the group consisting of SEQ ID NO: 2, SEQ ID NO: 3, and variants thereof; and a peptide tag and a peptide binding partner derived from an isopeptide protein. The recombinant Bm86Dock module elicits an immune response in the subject.

The recombinant Bm86Dock module of any preceding embodiment, can include at least one immunogenic fragment consisting of SEQ ID NO: 1.

The peptide tag and the peptide binding partner of any of the preceding embodiments, are selected from the group consisting of SpyTag/SpyCatcher, SpyCatcherΔN1, SpyCatcherΔN2, SpyCatcherΔN3, SpyCatcherΔN1C1, SpyCatcherΔN1C2, SpyTag002/SpyCatcher002, SpyTag003/SpyCatcher003, SdyTag/SdyCatcher, DogTag/DogCatcher, SnoopTag/SnoopCatcher and SilkTag/SilkCatcher.

The peptide tag of any of the preceding embodiments includes the amino sequence having at least 90%, at least 95% or 100% sequence identity with SEQ ID NO: 4.

The peptide binding partner of any of the preceding embodiments can include the amino sequence having at least 90%, at least 95% or 100% sequence identity with SEQ ID NO: 7.

Also provided herein is an expression vector that includes the recombinant Bm86Dock module or the Bm86Dock immunogen of any of the preceding embodiments.

Further provided herein is an immunogenic Bm86Dock-nanoparticle (Bm86Dock-NP) that includes: a self-assembling multimeric protein scaffold composed of a plurality of polypeptide subunits, and a plurality of immunogens. Each immunogen is conjugated to a polypeptide subunit of the plurality of polypeptide subunits. Each immunogen of the plurality of immunogens comprises a recombinant module (Bm86Dock) of a protein Bm86 including an amino acid sequence selected from the group consisting of SEQ ID NO: 2, SEQ ID NO: 3, and variants thereof. The Bm86Dock-NP elicits an immune response in the subject.

The immunogenic Bm86Dock-NP of the preceding embodiment, characterized in that an immunogen or at least one of a plurality of immunogens is displayed on the outer surface of the self-assembling multimeric protein scaffold.

The immunogenic Bm86Dock-NP of any of the preceding embodiments, characterized in that the immunogen displays at least one linear epitope and/or at least one conformational epitope of the native Bm86Dock module.

The immunogenic Bm86Dock-NP of the preceding embodiments, characterized in that the at least one linear epitope and/or the at least one conformational epitope is recognized by protective or neutralizing antibodies.

The immunogenic Bm86Dock-NP of any of the preceding embodiments, characterized in that the self-assembling multimeric protein scaffold has an icosahedral symmetry, a dodecahedral symmetry, an annular shape, or a cylindrical shape.

The immunogenetic Bm86Dock-NP of any of the preceding embodiments, characterized in that the polypeptide subunit is derived from a self-assembling multimeric protein of the group including of lumazine synthase (LS), ferritin, HCV envelope glycoprotein E2, Brucella outer membrane protein BP26, (3-annulus peptide, AP205, encapsulin, sHSP, CCMV, bacteriophage P22, 13-01, mutated I3-01 (mi3), I32-19, I32-58, and I53-50.

The immunogenic Bm86Dock-NP of any of the preceding embodiments, characterized in that the self-assembling multimeric protein scaffold is composed of at least 3, at least 4, at least 5, at least 6, at least 7, at least 8, at least 10, at least 12, at least 20, at least 24, at least 60, at least 120, at least 180, at least 240 polypeptide subunits. In some embodiments, the self-assembling multimeric protein scaffold is composed of 24 polypeptides subunits. In some other embodiments, the self-assembling multimeric protein scaffold is composed of 60 polypeptides subunits.

The immunogenic Bm86Dock-NP of any of the preceding embodiments, characterized in that each immunogen is conjugated to its polypeptide subunit via an isopeptide bond formed between a tag peptide and a peptide binding partner.

The immunogenic Bm86Dock-NP of the preceding embodiments, characterized in that the tag peptide is fused to each immunogen and the peptide binding partner is fused to each polypeptide subunit of the self-assembling multimeric protein scaffold.

The immunogenic Bm86Dock-NP of the preceding embodiments, characterized in that the tag peptide is fused to each polypeptide subunit of the self-assembling multimeric protein scaffold and the peptide binding partner is fused to each immunogen.

The immunogenic Bm86Dock-NP of any of the preceding embodiments, characterized in that the peptide tag/peptide binding partner is selected from the group including SpyTag/SpyCatcher, SpyCatcherΔN1, SpyCatcherΔN2, SpyCatcherΔN3, SpyCatcherΔN1C1, SpyCatcherΔN1C2, SpyTag002/SpyCatcher002, SpyTag003/SpyCatcher003, SdyTag/SdyCatcher, DogTag/DogCatcher, SnoopTag/SnoopCatcher and SilkTag/SilkCatcher.

The immunogenic Bm86Dock-NP of any of the preceding embodiments, characterized in that the peptide tag comprises the amino sequence having at least 90%, at least 95% or 100% sequence identity with SEQ ID NO: 4.

The immunogenic Bm86Dock-NP of any of the preceding embodiments, characterized in that the peptide binding partner includes the amino sequence having at least 90%, at least 95% or 100% sequence identity with SEQ ID NO: 7.

The immunogenic Bm86Dock-NP of any of the preceding embodiments, characterized in that each immunogen of the plurality of immunogens includes an amino acid sequence selected from the group consisting of SEQ ID NO: 2, SEQ ID NO: 3, and variants thereof.

The immunogenic Bm86Dock-NP of any of the preceding embodiments, characterized in that each polypeptide subunit of the plurality of polypeptide subunits includes an amino acid sequence selected from the group consisting of SEQ ID NO: 8, SEQ ID NO: 9, and variants thereof.

The immunogenic Bm86Dock-NP of any of the preceding embodiments, characterized in that each immunogen which includes an amino acid sequence selected from the group consisting of SEQ ID NO: 2, SEQ ID NO: 3, and variants thereof, is conjugated with each polypeptide subunit which includes an amino acid sequence having at least 90%, at least 95% or 100% sequence identity with SEQ ID NO: 7 via an isopeptide bond.

The immunogenic Bm86Dock-NP of any of the preceding embodiments, characterized in that each immunogen includes an amino acid sequence is selected from the group consisting of SEQ ID NO: 2, SEQ ID NO: 3, SEQ ID NO: 5, SEQ ID NO: 6, and variants thereof, is conjugated with each polypeptide subunit which includes an amino acid sequence having at least 90%, at least 95% or 100% sequence identity with SEQ ID NO: 8 or SEQ ID NO: 9 via an isopeptide bond.

The immunogenic Bm86Dock-NP of any of the preceding embodiments, characterized in that each immunogen includes an amino acid sequence is selected from the group consisting of SEQ ID NO: 5, SEQ ID NO: 6, and variants thereof, is conjugated with each polypeptide subunit which includes an amino acid sequence having at least 90%, at least 95% or 100% sequence identity with SEQ ID NO: 8 via an isopeptide bond.

A method for producing the immunogenic Bm86Dock-NP of any of the preceding embodiments including the steps of: expressing the plurality of immunogens of any of the embodiments in a prokaryotic or eukaryotic expression system, independently expressing the plurality of polypeptide subunits in a prokaryotic or eukaryotic expression system, conjugating said plurality of polypeptide subunits with said plurality of immunogens via an isopeptide bond; and allowing said plurality of polypeptide subunits conjugated to said plurality of immunogens to self-assemble into the self-assembling multimeric protein scaffold such as to display said plurality of immunogens on the outer surface of said immunogenic Bm86Dock-NP.

The method of the preceding embodiment, characterized in that conjugating of said plurality of polypeptide subunits with said plurality of immunogens is done at ratios between 1:0.5 and 1:3. For example, the ratio may be 1:0.5, 1:1, 1:1.5, 1:2 or 1:3.

Provided herein is an immunogenic composition including at least one Bm86Dock immunogen of any of the preceding embodiments or at least one immunogenic Bm86Dock-NP of any of the preceding embodiments or at least one immunogenic Bm86Dock-NP produced according to the method of any of the preceding embodiments and a pharmaceutically acceptable carrier and/or at least one adjuvant.

The immunogenic composition of the preceding embodiments, characterized in that the at least one adjuvant is selected from aluminum-containing adjuvants, liposome-based adjuvants, Emulsigen range of adjuvants, the Montanide Gel, Montanide ISA O/W and the Montanide range of adjuvants, Freund's complete and incomplete adjuvant, Water-in-Oil emulsions and Oil-in-Water emulsions such as MF59.

The immunogenic composition of the preceding embodiments, wherein the aluminum-containing adjuvant is aluminum oxyhydroxide-based hydrogel (AlOH).

Further disclosed herein is the use of the immunogenic composition of any of the preceding embodiments as a medicament, preferably as a vaccine.

The immunogenic composition of any of the preceding embodiments is for use in the treatment or the prevention of a tick infestation and the transmission of a tick-borne pathogen in a subject.

The immune composition of the preceding embodiments is characterized in that the a tick-borne disease is selected from the group including Babesiosis, Anaplasmosis, Cytauxzoonosis, Ehrlichiosis, Lyme disease, Southern Tick-Associated Rash Illness, Rickettsioses, Tick-Borne Relapsing Fever, Borrelia mayonii Disease, Typhus, Tularemia, Tick-Borne encephalitis, Powassan Virus Disease, Colorado Tick Fever, Heartland virus, Bourbon virus, Kyasanur forest disease, Omsk Hemorrhagic Fever, Crimean-Congo Hemorrhagic Fever, Severe Febrile Illness, Tick paralysis and Alpha-gal Syndrome.

Provided herein is a method for treating or preventing a tick infestation and the transmission of a tick-borne pathogen in a subject including administering an effective amount of the immunogenic composition of any of the preceding embodiments.

The method of the preceding embodiment is characterized in that the immunogenic composition is administered intramuscularly, subcutaneously or orally.

The method of any of the preceding embodiments, is characterized in that the immunogenic composition is administered as a prime series of at least 2 doses about 3 to 4 weeks apart followed by a booster series of between 1 to 4 doses each administered 4 months, 6 months to 1 year apart.

The method of any of the preceding embodiments is characterized in that the subject is a domestic mammal, a livestock, or a bird.

Provided herein is a vaccine formulation comprising 5 to 200 μg of Bm86Dock-NP of any of the preceding embodiments or Bm86Dock of any of the preceding embodiments, and 15% V/V of Montanide Gel 01 PR in storage buffer, or Tris.

Provided herein is a vaccine formulation comprising 5 to 200 μg of the immunogen Bm86Dock of any one of claims 1-2, or the immunogenic Bm86Dock-NP of any of the preceding embodiments and an aluminum oxyhydroxide-based hydrogel (AlOH) in storage buffer, or Tris.

Further provided herein is a method for treating or preventing tick infestations in mammals or birds that includes the intramuscular administration of a vaccine comprising a Bm86Dock module having the amino acid sequence selected from the group consisting of SEQ ID NO: 2, SEQ ID NO: 3, and variants as a prime series approximately 3 to 4 weeks apart followed by a booster series approximately 4, 6 months to 1 year apart.

### DESCRIPTION OF THE DRAWINGS

FIGS. 1A-E are representations of Bm86 and module Bm86Dock. A. Scheme showing the molecular architecture of the Bm86 protein derived from R. microplus. B. Cartoon representation of predicted 3D structure of Bm86 (Uniprot code P20736) showing Bm86Dock in black. C. Prediction quality of Bm86Dock structure colored according to AlphaFold2 score (pLDDT). D. Structural alignment of Bm86Dock (black solid cartoon) against a SEA domain (white transparent cartoon). E. Topology of Bm86Dock module. Beta-sheets are named A to G, while alpha-helix are numbered 1 and 2. Structural elements are colored according to the belonging exposed surface. Disulfide bonds are indicated by dashed lines, glycosylation sites are pointed out by arrow tails, involved residues are highlighted in bold. The N and C terminals are indicated.

FIGS. 2A-2C show the phylogenetic variation of the Bm86Dock module. A. Sequence identity conservation between all available non-redundant variants of Bm86Dock from R. microplus at GenBank gathered around the world and the reference sequence from Australia (sequence #47, GenBank accession M29321 [Rand et al., Cloning and expression of a protective antigen from the cattle tick Boophilus microplus., Proc. Natl. Acad. Sci. 86, 9657-9661 (1989)]). Each global and by-residue sequence identity is indicated. Preserved residues are colored black, while non-equal residues are colored white. Identified topological motifs in the structure are mapped on the sequence at the bottom. L1 is the loop between β1 and α1. Motifs are grouped according to the displayed immunogenic surface on the protein. B. Mapping of mean sequence identity by-residue in the 3D structure of Bm86Dock. The less conserved residues are labeled and represented by sticks in the corresponding color scale. C. Phylogenetic tree of Bm86Dock according to sequence identity and comprising both Bm86-like and ATAQ-like protein homologs. The multiple sequence alignment is based on a structural fitting by the STAMP algorithm. Structures for each sequence were retrieved from the Alphafold2 database. The structural similarity between Bm86-like or ATAQ-like proteins is shown.

FIGS. 3A-3C show the expression and purification of Bm86Dock from E. coli and Mammalian cells. A. SDS PAGE gel showing the periplasmic and cytoplasmic expression of Bm86Dock in E. coli. B. SDS-PAGE gel showing the complete purification pipeline of Trx-Bm86Dock and size exclusion chromatography. C. Expression and purification of Bm86Dock in mammal cells.

FIGS. 4A-4C show the characterization of predicted disulfide bonds of Bm86Dock expressed in E. coli. A. Schematic representation of the Bm86Dock sequence with the cysteines in bold and the two disulfide bonds indicated with lines (SEQ ID NOs: 12-13). The disulfide bonds are expected to be intrapeptide or interpeptide according to the enzymatic digestion. The first two residues of the sequence in gray (Glycine and Serine) remained after the TEV cleavage. B. The observed sequence coverage by mass spectral analysis is indicated by the underlined amino acids (SEQ ID NOs: 14-16). The presence of the expected intrapeptide and interpeptide disulfide bonds is indicated in the spectrum. Both disulfide bonds were confirmed by peptide fragmentation. C. Identification of the interpeptide disulfide bond by peptide fragmentation. Several fragments of the expected mass for a peptide containing the disulfide bond were obtained and are indicated in the spectrum (SEQ ID NOs: 17-19).

FIGS. 5A-5C show schematic representation of Bm86Dock immunogenic nanoparticle (Bm86Dock-NP).

FIG. 6. shows the covalent coupling of Bm86Dock or Bm86Dock002 to a protein nanoparticle (NP) via in-vitro ligation methods.

FIGS. 7A-7C show the structural and biophysical characterization of Bm86Dock and Bm86Dock002. A. Circular dichroism spectra of Bm86Dock and Bm86Dock002 in water. B. Circular dichroism spectra of Bm86Dock in water adding increasing concentrations of urea. C. Thermal unfolding curves of Bm86Dock and Bm86Dock002 measured by nanoDSF.

FIG. 8 shows a graph comparing IgG titers after immunization with Bm86Dock and Bm86Dock002.

FIG. 9 shows a graph comparing IgG titers after immunization with Bm86Dock-NP vaccine formulated with different adjuvants.

FIG. 10 provides graphs demonstrating the immunogenicity of Bm86Dock or Bm86Dock-NP in bovine animals.

FIGS. 11A-11D show the field efficacy test evaluating the immunization with Bm86Dock-NP vaccine. A. Scheme of the experimental design. B. IgG antibody titer in animals immunized with Bm86Dock-NP. C. IgG antibody titer in control animals. D. Measured efficacy parameters in control and vaccinated animals (pool and selection).

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be used and other methods developed without departing from the broad concepts of the current invention. All references cited anywhere in this specification, including the Background and Detailed Description sections, are incorporated by reference as if each had been individually incorporated.

Some definitions are included herein for the purpose of understanding the present subject matter and the appended claims. The abbreviations used herein have their conventional meanings within the chemical and biological arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The present description identifies certain amino acid sequences (peptides or polypeptides) as part of the invention. It is to be understood that the specifically identified sequences adequately describe other sequences that contain less than 100% sequence identity to the identified sequences while providing the same function and/or a similar 3D structure. For example, a polypeptide may contain less than 100% sequence identity to a polypeptide specifically identified herein while providing the same function and a 3D structure similar to that of the polypeptide in its native conformation. For example, a polypeptide may have 90%, 91%, 92%, 93%, 94%, 95%, 96%, 97%, 98%, sequence identity or 99% sequence identity with a polypeptide specifically disclosed herein and still retain the same or sufficiently similar activity, functionality and/or 3D structure as the specifically identified immunogenic polypeptide.

As used herein, the term “variants” refers to stable modified Bm86Dock polypeptide variants, examples of which are described herein. Variants may be generated with the assistance of a structure-based computational method. The aim is to alter the native sequence while retaining and/or enhancing desirable properties (e.g., folding, stability). In such variants, amino acid substitutions leading to conservative substitutions or changes at core residues may be introduced in SEQ ID NO: 2 or SEQ ID NO: 5. Core residues may be defined as amino acids that are not surface residues. Surface residues may be defined as residues that may form epitopes and/or may be involved in interactions with other proteins. Mutations may be introduced by standard techniques, such as site-directed mutagenesis and PCR-mediated mutagenesis. In some embodiments, Bm86Dock variants may have amino acid sequences having at least 90%, at least 95% or at least 99% identity with SEQ ID NO: 2 or SEQ ID NO: 5. For example, the variant Bm86Dock002 comprising the amino acid sequence SEQ ID NO: 3 or SEQ ID NO: 6 has 95% of sequence identity with SEQ ID NO: 2 and SEQ ID NO: 5, respectively. Variant Bm86Dock002 carries 5 mutations in core residues outside SEQ ID NO: 1, exhibits improved thermal stability while retaining the 3D structure of native Bm86Dock.

As used herein, the term “epitope” refers to an antigenic determinant, a portion of the Bm86Dock module that is recognized by the immune system in context of the invention, specifically by antibodies, B cells, or T cells. An Epitope may be a B-cell epitope or a T-cell epitope. A B-cell epitope is a peptide sequence which is required for recognition by specific antibody producing B-cells. For example, a B-cell epitope refers to a specific region of Bm86Dock that is recognized by an antibody. An epitope may be a conformational epitope or a linear epitope. A linear or continuous epitope is defined by the primary amino acid sequence of a particular region of a Bm86Dock. A conformational epitope is defined by the conformational structure of the native Bm86Dock. A conformational epitope may be continuous or discontinuous, i.e., components of the epitope can be situated on disparate parts of the Bm86Dock, which are brought close to each other in the folded native structure.

As used herein, the term “fragment” refers to a peptide or polypeptide of chain-type polymer formed by at least 6 amino acid residues which are linked to each other via peptide bonds. It may include amino acid sequences that are conservative variations. The terms fragment, peptide and polypeptide are used interchangeably. In the context of the invention, an immunogenic fragment may have one or more linear and/or conformational epitopes that can induce a humoral and/or cell-mediated immune response. For example, an immunogenic fragment of Bm86Dock can be the peptide SEQ ID NO: 1. It is understood that Bm86Dock may comprise more than one immunogenic fragment and may display additional epitopes.

As used herein, the term “antibody” refers to a protein produced by the B-cells of the immune system that can identify, bind and neutralize an antigen e.g., SEQ ID NO: 1 of Bm86Dock module. The antibody may have neutralizing properties and may be capable of conferring protective immunity against ticks and the transmission of tick-borne pathogens.

As used herein, the term “domain” refers to any region in a protein showing a particular functional characteristic or structural feature.

As used herein, the term “module” refers to a domain or subdomain of a native protein which can fold independently while retaining its native 3D structure. That is, the same or sufficiently similar 3D structure as the 3D structure of the polypeptide within the native full-length protein.

As used herein, the term “native” refers to the 3D structure of a protein or module in its natural biologically active state. The structure is determined by the sequence of amino acids of the protein or module, as well as any post-translational modifications. A native module can carry out its designated function independently of the native protein it derives from.

As used herein, the term “isopeptide bond” refers to an amide bond between an amino group of one amino acid and a carboxyl group of another amino acid in which at least one of these groups is not attached to the α-carbon. The most common isopeptide bonds are formed between the ε-amino group of a lysine residue in one protein and a carboxyl group in a second protein. A “peptide tag” is a peptide that forms a spontaneous covalent isopeptide bond, upon binding with its “peptide binder partner”. An “isopeptide protein” refers to a coupled peptide tag/peptide binder partner able to spontaneously form an isopeptide bond.

Several of the most efficacious recombinant vaccines against ticks contain or are based on the Bm86 protein. In particular, the recombinant full-length protein Bm86 (WO1988003929A1) and related homologs Bm95, Bm86TX (US20180085443A1), Bm86Uy (BR102015017673B1) have been used alone, in cocktails like Bm86+pP0 (EP2623114B1), Bm86Uy+Bm05 (BR102015017673B1), Bm86+Subolesin (WO2014154847A1) or as chimeric fusions Bm95-MSP1a (U.S. Pat. No. 8,435,537B2), Bm86-pP0 (EP2623114B1) for producing subunit tick vaccines. Several chimeric peptides generated from small non-consecutive sequences (<20 amino acids) of Bm86 are also patented, such as SBm4912 and SBm7462 (EP1289545B1), and the tandem SBm7462 (WO2015054764A1).

Development of recombinant tick vaccines based on Bm86 has been hampered by production challenges and retention of efficacy. The effectiveness of expression systems based on low-cost bacteria are limited by the length of the protein to be produced and the presence of hydrophobic patches as well as disulfide bonds which lead to antigen aggregates. Antigen aggregates of poor quality may require re-solubilized from inclusion bodies, adding steps to the manufacture process. Moreover, prior chimeric vaccines show varying degrees of effectiveness ranging from ˜50% to ˜90%. due to the genetic variability within populations and within species. Differences as low as 3% in sequence identity may be enough to diminish vaccine efficacy to unacceptable levels (e.g., weak protective immune response). These structural features may also prevent the full-length sequence from being coupled to nanoparticle display technologies. As a result, only two vaccines based on the Bm86 protein are commercialized: TickGARD® and Gavac®. Some domains derived from Bm86 such as sequence-predicted EGF-like domains from N and C-terminal regions of Ixodes scapularis were evaluated as vaccine candidates but did not elicit durable or potent immunity. The EGF-like sequences encode for domains rather than modules because they were expressed as inclusion bodies requiring urea to solubilize them as denatured polypeptides. The present disclosure identifies chimeric proteins, composition, and vaccines that overcome challenges of the art, and methods allowing for the production of vaccines that can be varied in a manner to maintain safety, stability and efficacy.

As identified herein, an effective way to address the limited effectiveness of Bm86 vaccine is the use of self-assembling multimeric protein-based nanoparticles which may display multiple copies of Bm86-derived immunogens. In some aspects of the embodiments, the rational design of recombinant Bm86Dock modules which display Bm86-derived epitopes of the R. microplus Bm86 protein results in potent immunogens capable of inducing a protective immune response and can be active across a broad spectrum of tick species.

Methods for producing the tick vaccines in either E. coli or mammal cells are also improved, yielding soluble and homogeneous vaccines in a cost-effective manner, while reducing the batch-to-batch variability.

### Bm86Dock Module

Bm86Dock is a newly discovered functional folding module which has never been used before the present invention as an immunogen in vaccine formulations against ticks. Bm86 is a multidomain glycoprotein of about 650 residues, which is present in the midgut of ticks [Rand et al., Cloning and expression of a protective antigen from the cattle tick Boophilus microplus., Proc. Natl. Acad. Sci. 86, 9657-9661 (1989)]. Bm86 has proven immunogenicity and a role in the transmission of bacterial pathogens from infected ticks to hosts [Pereira et al., Rhipicephalus microplus: An overview of vaccine antigens against the cattle tick, Ticks Tick-Borne Dis. 13, 101828 (2022); Koci et al., Antibodies against EGF-like domains in Ixodes scapularis BM86 orthologs impact tick feeding and survival of Borrelia burgdorferi, Sci. Rep. 11, 6095 (2021)]. Closely related homolog proteins named ATAQ are also present in ticks; however, their expression pattern differs in tissue localization and developmental stage [Nijhof et al., Bm86 homologues and novel ATAQ proteins with multiple epidermal growth factor (EGF)-like domains from hard and soft ticks, Int. J. Parasitol. 40, 1587-1597 (2010)].

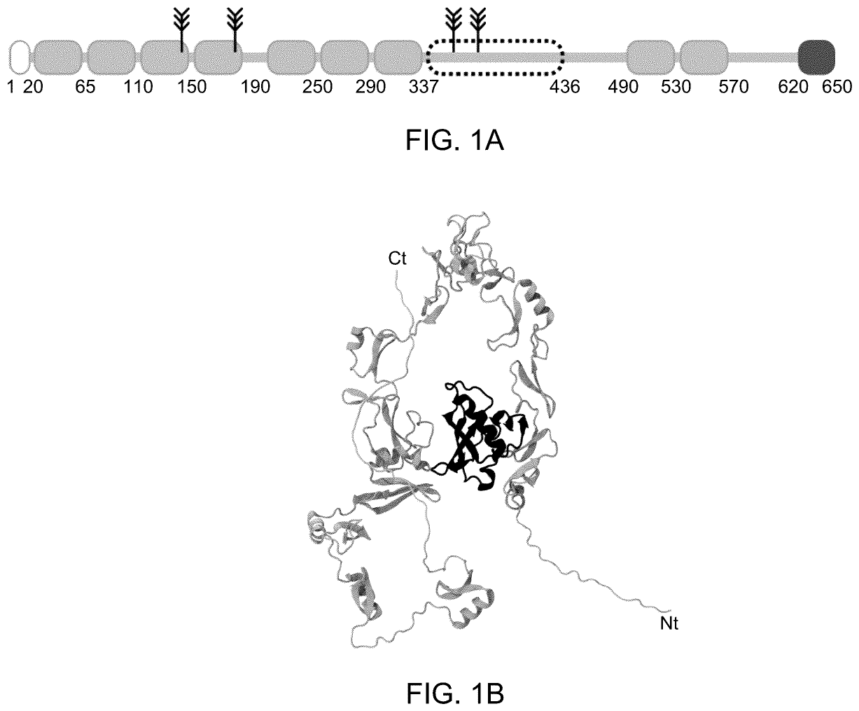

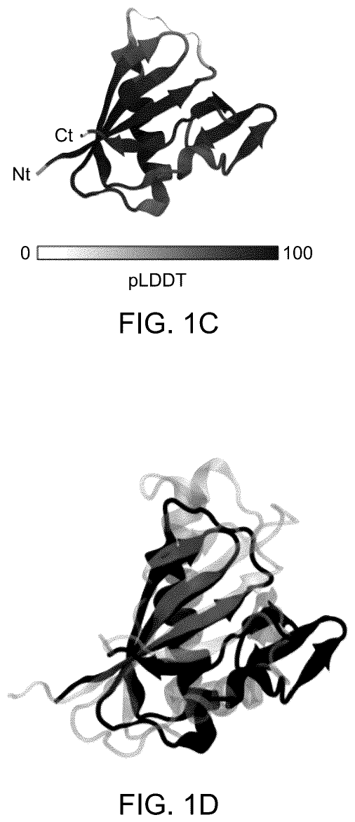

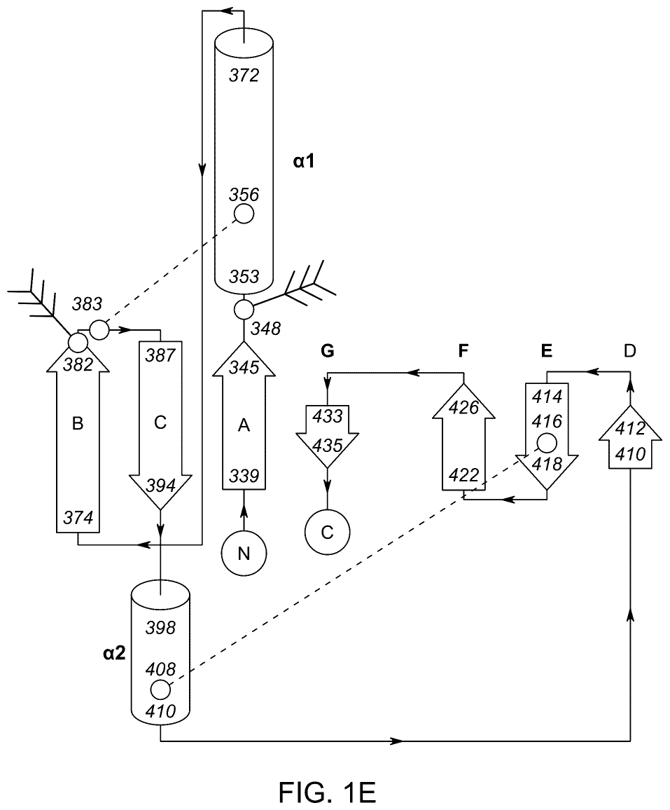

The molecular architecture of the Bm86 protein family has been predicted through sequence homology to known functional domains, since an experimentally validated structure is not yet available (FIG. 1A). The general structure contains a 20-residue long N-terminal signal peptide, followed by a tandem of seven variable EGF-like domains spanning up to approximately 350 residues of the full-length sequence. Glycosyl-Phosphatidylinositol anchor, transmembrane or EGF-like domains are found from residue about 480 to the end of the sequence. To date, no other structural domains have been described, nor reported in the Pfam database [Mistry et al., Pfam: The protein families database in 2021, Nucleic Acids Res. 49, D412-D419 (2021)].

In some embodiments, a Bm86Dock module (also referred to herein as simply Bm86Dock or recombinant Bm86Dock or the variants described herein) is derived from full-length Bm86 which was identified based on the predicted 3D structure of Bm86 retrieved from the Alphafold2 database [Varadi et al., AlphaFold Protein Structure Database: massively expanding the structural coverage of protein-sequence space with high-accuracy models, Nucleic Acids Res. 50, D439-D444 (2022)](FIG. 1B). The novel Bm86Dock module was identified and selected based on predicted structural, biochemical, biological and immunological features to optimize the protein expression and its potential immunogenicity for vaccine formulations. The new uncharacterized folding Bm86Dock module includes one of the amino acid sequences SEQ ID NO: 2. (FIG. 1B) Structurally, Bm86Dock is a small globular protein module (<20 kDa) having a high-quality prediction score (FIG. 1C), few hydrophobic patches and only two predicted disulfide bonds. In contrast, the full-length Bm86 protein is 89 kDa and has 65 cysteines. In addition, Bm86Dock has 2 putative glycosylation sites at Asparagine 348 and 382, which may increase its solubility and immunogenicity. Notably, Bm86Dock has no more than 30% of sequence identity with host proteins or non-tick proteins (NCBI blast) which reduces the risk of antibodies cross reactivity. To further characterize the Bm86Dock module, a search for structural homologs with Dali was performed against the Protein Data Bank (PDB) [Holm, Dali server: structural unification of protein families, Nucleic Acids Res. 50, W210-W215 (2022); Berman et al., The Protein Data Bank, Nucleic Acids Res. 28, 235-242 (2000)]. The SEA domain was identified as the most structurally relevant domain with a percentage of identity of 11% (Z-score of 7.2 against PDB id. 1IVZ, FIG. 1D). Bm86Dock is a new functional folding module and its topology shows a non-trivial arrangement of alpha-helix and beta-sheet motifs defining 4 solvent exposed surfaces (FIG. 1E).

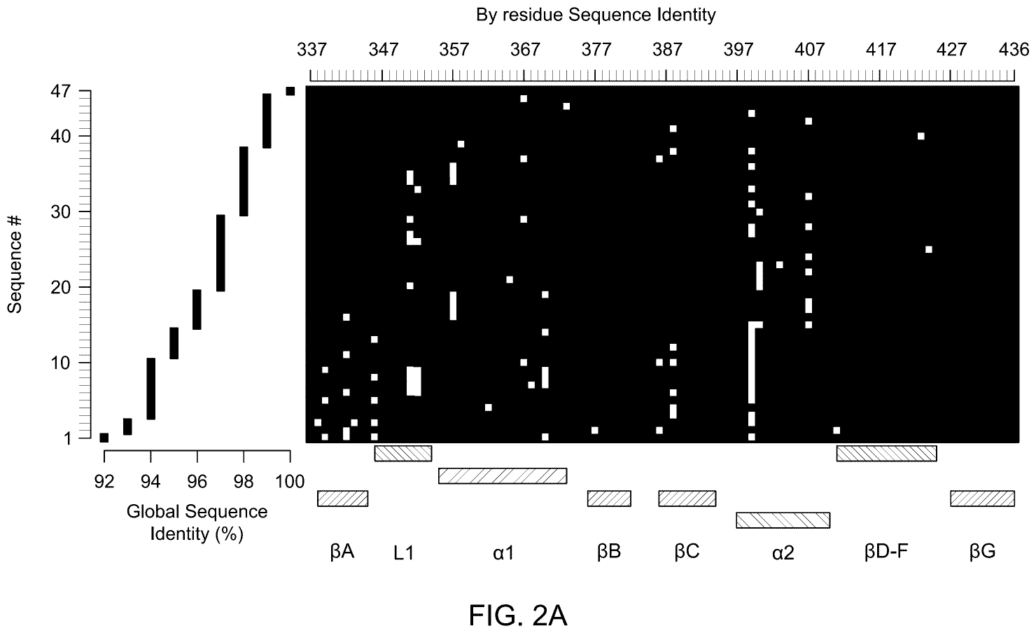

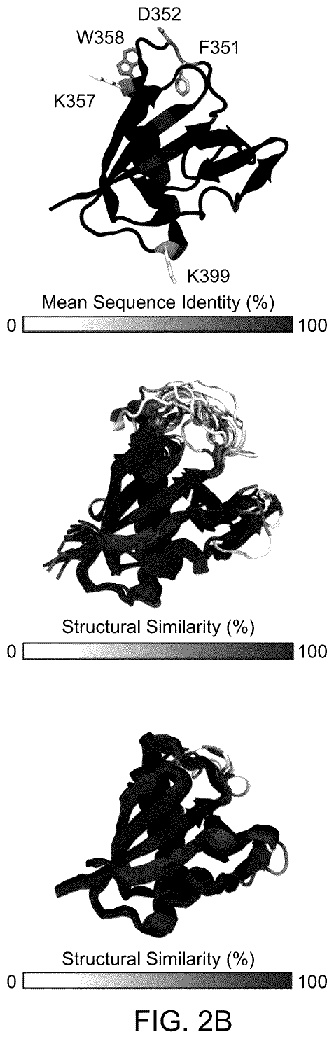

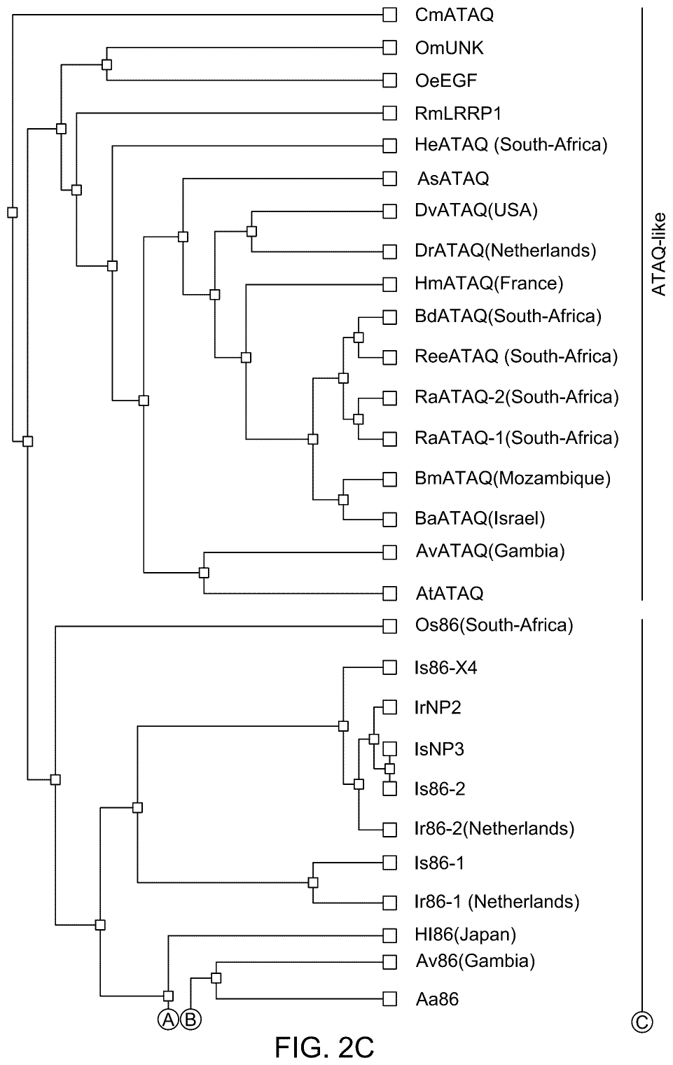

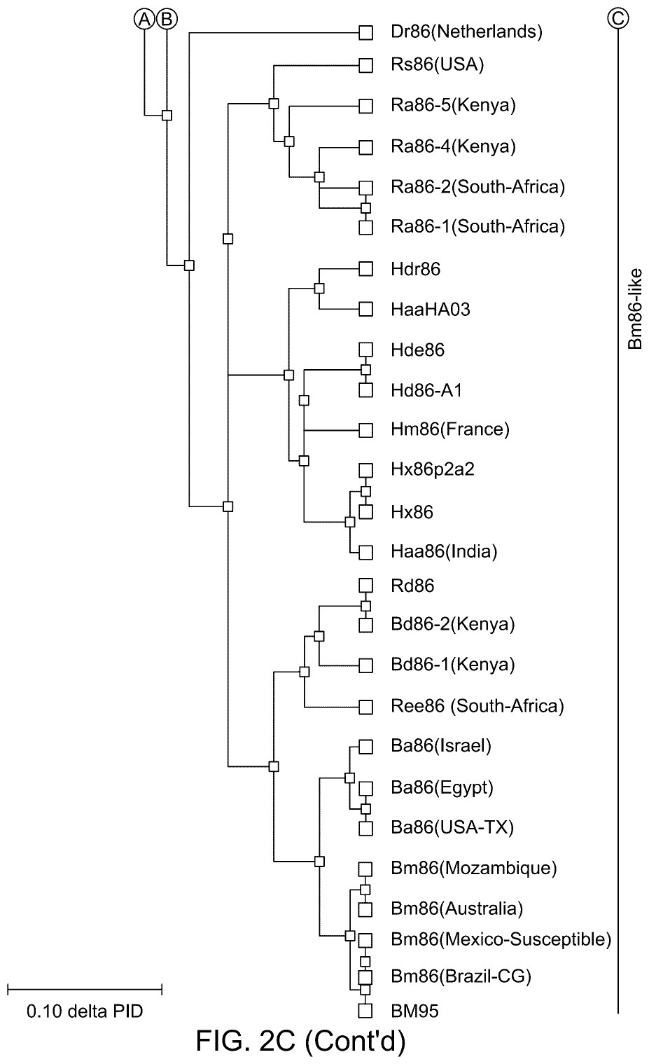

Phylogenetic analysis of variants of Bm86Dock from R. microplus available at GenBank shows a high global and by-residue sequence identity conservation (FIG. 2A). Indeed, most of the exposed surfaces were conserved, while only a few residues changed in the average population. (FIG. 2B) The less conserved residues in Bm86Dock were located at the beginning of alpha helix 1 and 2. (FIGS. 2A-2B) Of these 5 residues, Phenylalanine 351 was not solvent exposed according to the 3D model and may not be involved with an immunogenic epitope. Further analysis showed that Bm86Dock has high sequence homologies and structure conservation among different tick species. (FIG. 2C) Indeed, Bm86Dock was a fingerprint for Bm86-like and ATAQ-like proteins, being able to accurately reproduce the phylogenetic tree of the full-length sequence.

In the context of the invention, Bm86Dock is a small well folded soluble module, making it suitable for expression as a single or fused polypeptide as well as for covalent coupling with other proteins by various in-vitro ligation systems.

In some embodiments, the Bm86Dock module and its variants carry at least one immunogenic fragment of 14 amino acids as defined in SEQ ID NO: 1, located in the alpha-helix 2. The immunogenic fragment was previously included in chimeric peptide vaccines SBm4912 and SBm7462 [Patarroyo et al., Immunization of cattle with synthetic peptides derived from the Boophilus microplus gut protein (Bm86), Vet. Immunol. Immunopathol. 88, 163-172 (2002)]. Within the Bm86Dock module, SEQ ID NO: 1 is displayed in its native structural context, allowing for both its recognition by T-cells and antibodies produced by B-cells, a feature hardly met by peptide vaccines.

In some embodiments, the Bm86Dock module and its variants, the including Bm86Dock002 module described herein display additional conserved regions with potential linear and/or conformational epitopes, making it less susceptible for small sequence variations and reducing the risk for mutational scape. Notably, Bm86Dock module and its variants have no host homologs, allowing the production of broad-spectrum vaccines against different tick species while granting specificity and safety. It should be noted that other domains of Bm86 outside Bm86Dock were tested for their immunogenicity without showing high potency [Koci et al., Antibodies against EGF-like domains in Ixodes scapularis BM86 orthologs impact tick feeding and survival of Borrelia burgdorferi, Sci. Rep. 11, 6095 (2021)].

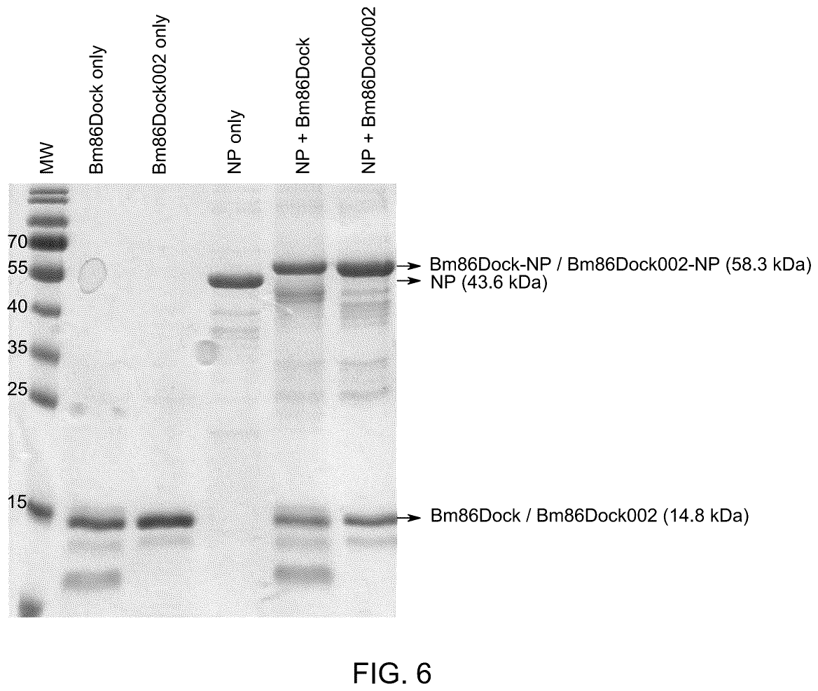

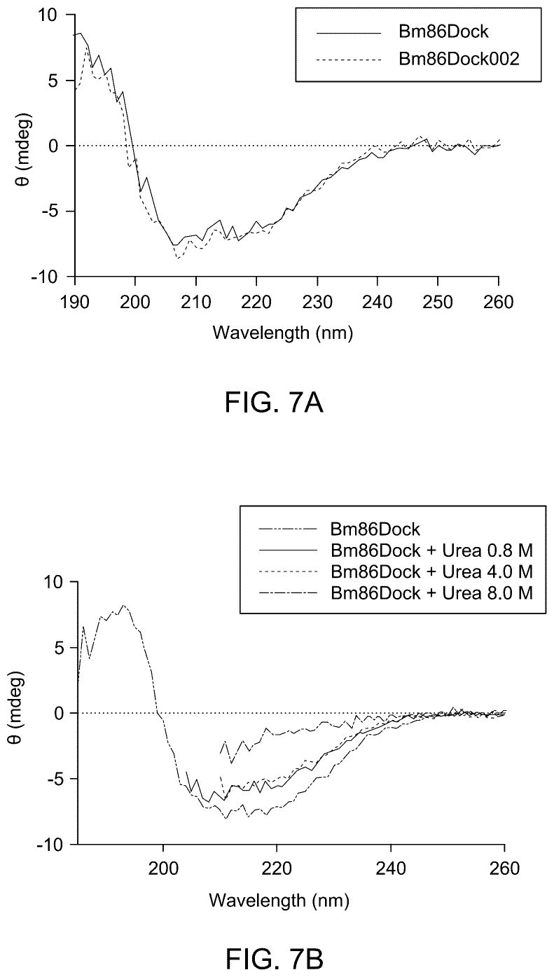

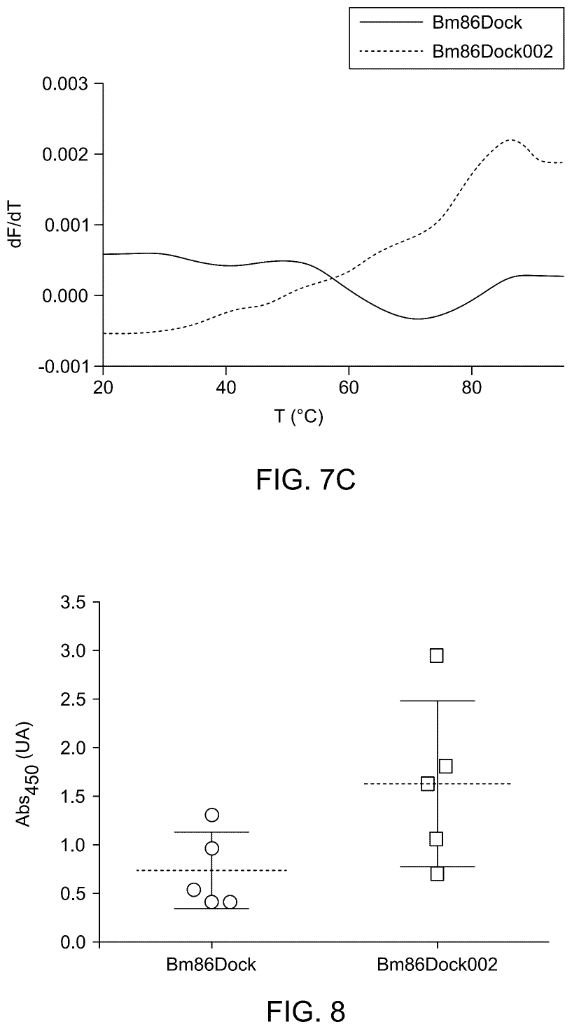

In other aspects of the embodiments, one or more amino acid substitutions leading to conservative substitutions and/or changes at core residues may be introduced in the sequence of the Bm86Dock module. Structurally, Bm86Dock variants may improve thermal stability while adopting a 3D structure similar to that of native Bm86Dock. Functionally, Bm86Dock variants may induce a stronger immune response while conserving the same surface residues involved in linear and/or conformational epitopes of native Bm86Dock. An exemplary embodiment of a Bm86Dock variant is described herein as Bm86Dock002 having the amino acid sequence SEQ ID NO: 3. Bm86Dock002 has improved thermal stability (FIG. 7C) and immunogenicity (FIG. 8) compared to Bm86Dock, while retaining the 3D structure of native Bm86Dock (i.e., conformational epitopes are conserved) (FIG. 7A). Bm86Dock002 has 95% sequence identity with the sequence of native Bm86Dock and differs by 5 substitutions of core residues (i.e., non-exposed and non-immunogenic residues). The substitutions are outside the immunogenic fragment (SEQ ID NO: 1) and are not present alone or in any combination in any known natural variant of R. microplus presented in FIG. 2A. Bm86Dock002 exhibits the same versatility as Bm86Dock and may be fused to a peptide tag or a peptide binding partner (FIG. 6)

Additional Bm86Dock variants may be constructed in a similar manner as Bm86Dock002. The Bm86Dock variants do not carry mutation(s) in surface residues that may form conformational an/or linear epitopes. The mutations are limited to the core residues of the Bm86Dock; such mutations improve the thermal stability of the variants without disrupting the 3D structure allowing the display of epitopes. For example, variants may be generated to provide substitutions similar to those exemplified in SEQ ID NO: 3. Mutation of surface residues that may form conformational an/or linear epitopes are avoided. However, the substitutions in SEQ ID NO: 3 may be modified by selecting different or additional core residues proximal to those changed in SEQ ID NO: 3 and substituting other amino acids that do not disrupt the 3D structure, i.e., maintain the display of epitopes.

Bm86Dock and the variants as described herein are potent immunogens. The 3D structure of Bm86Dock and its variants facilitate the display of multiple highly immunogenic conformational and/or linear epitopes, which elicit the production of a high-quality antibody repertoire. That is an advantage against full length or peptide vaccines, such as Bm86 (WO1988003929A1), Bm86Uy (BR102015017673B1), SBm4912 and SBm7462 (EP1289545B1), and tandem SBm7462 (WO2015054764A1).

Bm86Dock and the variants described herein are safe. Because they are absent from the mammal proteome, autoimmune responses or antigen tolerance can be prevented in immunized mammals. In contrast, vaccines containing full-length Bm86 protein with EGF domains, for which homologs are expressed in mammals, result in reduced safety.

Bm86Dock and the variants described herein provide broad protection. Bm86Dock sequences and structures are largely conserved between different tick species. In contrast, the efficacy of the full-length protein vaccines, such as Bm86 vaccine described in WO1988003929A1 and Bm86Uy vaccine described in BR102015017673B1, depends on the circulating parasite due to immune responses towards variable immunodominant regions. Similarly, the presence of several conserved immunogenic regions in Bm86Dock makes it less susceptible to small sequence variations than peptide vaccines, including SBm4912 and SBm7462 disclosed in EP1289545B1, and tandem SBm7462 disclosed in WO2015054764A1.

Bm86Dock and the variants described herein are modular and adaptable. The size, solubility and capability to fuse adaptor proteins allow for a wide range of applications. The versatility of the immunogenic module is yet another advantage over the full-length Bm86 protein.

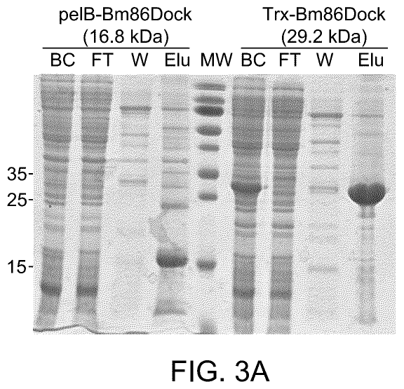

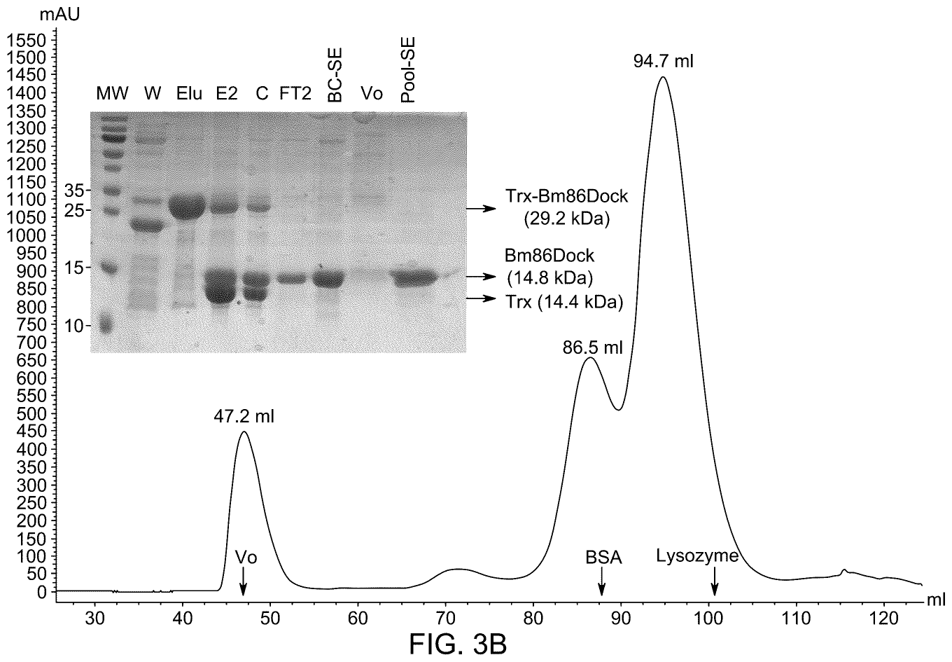

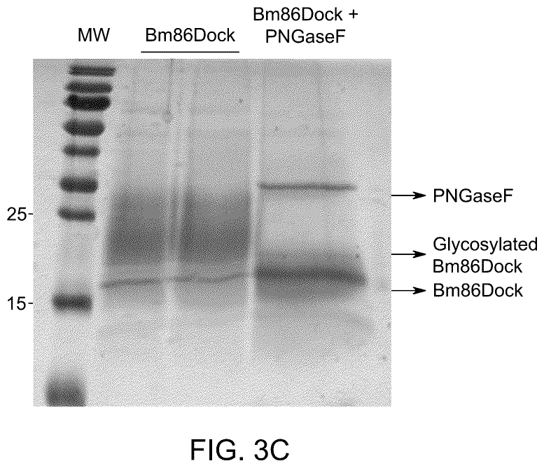

The production of Bm86Dock and the variants described herein is cost-effective. Bm86Dock and its variants may be produced in different expression systems including in E. coli and mammalian cells yielding high-quality proteins. (FIGS. 3A-3C) The methods of the embodiments are scalable, thus lowering costs and facilitating technology transfer. That is a significant advantage compared to synthetic peptide vaccines such as SBm4912 and SBm7462.

### Immunogenic Bm86Dock-Nanoparticles (Bm86Dock-NP)

The efficacy of a vaccine may rely on several factors including the structural design of the immunogen, the supplementation with adjuvants and potentially the immunization dosing regimen. The structural design of the immunogenic Bm86Dock module is especially critical to enhance the humoral response given that B-cells recognize antigens based on their shape and density on the surface of a pathogen. The display of multiple copies of the recombinant Bm86Dock module or its variants in a multivalent structure mimics the structure of the pathogen which potentially increases the activation of antigen-specific B cells through receptor crosslinking, leading to the production of neutralizing antibodies. (FIGS. 8-11)

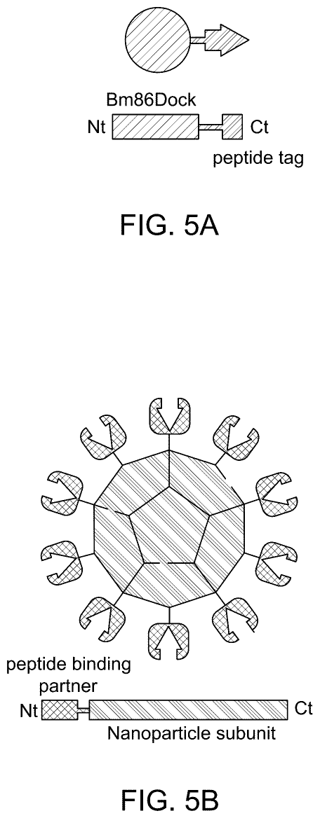

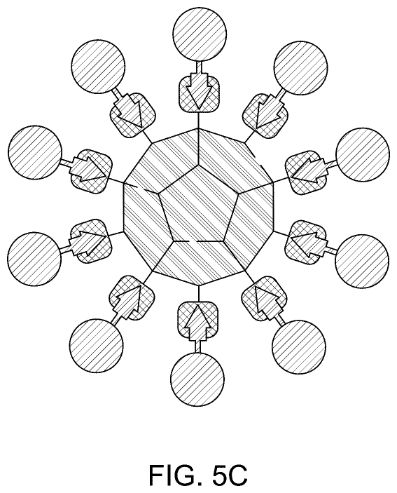

In some embodiments, Bm86Dock-NP includes a self-assembling multimeric protein scaffold composed of a plurality of polypeptide subunits, and a plurality of immunogens, wherein each immunogen is conjugated to a polypeptide subunit of the plurality of polypeptide subunits, and wherein each immunogen of the plurality of immunogens comprises a recombinant module (Bm86Dock) of a protein Bm86 comprising an amino acid sequence selected form the group consisting of SEQ ID NO: 2 and SEQ ID NO: 3. The Bm86Dock-NP elicits an immune response in the subject. Notably, the plurality of recombinant Bm86Dock display on the outer surface of the self-assembling multimeric protein scaffold. (FIGS. 5A-5C) For example, the self-assembling multimeric protein scaffold may be composed of at least 3, at least 4, at least 5, at least 6, at least 7, at least 8, at least 10, at least 12, at least 20, at least 24, at least 36, at least 48, at least 60, at least 120, at least 180, at least 240 polypeptide subunits. Preferably, the self-assembling multimeric protein scaffolds are derived from Ferritin or lumazine synthase (LS), and are composed of 24 polypeptide subunits or 60 polypeptide subunits.

In some aspects of the embodiments, the self-assembling multimeric protein scaffold may have an icosahedral symmetry, a dodecahedral symmetry, an annular shape, or a cylindrical shape.

In some aspects of the embodiments, Bm86Dock-NP displays at least one linear epitope and/or at least one conformational epitope of the native Bm86Dock module. A linear epitope may consist of continuous residues within the amino acid sequence of Bm86Dock module. On the other hand, a conformational epitope consists of residues that are discontinuous in the Bm86Dock sequence but come within proximity of each other to form an antigenic/immunogenic surface on the folded Bm86Dock module. Preferably a conformational epitope is displayed in the native 3D configuration of Bm86Dock. The recognition of epitopes on Bm86Dock-NP leads to the stimulation and activation of B-cells/T-cells and the subsequent production of protective or neutralizing antibodies. It is understood that the Bm86Dock variants do not carry mutation(s) in surface residues that may form conformational an/or linear epitopes. The mutations are limited to the core residues of the Bm86Dock as exemplified in SEQ ID NO: 3; such mutations improve the thermal stability of the variants without disrupting the 3D structure allowing the display of epitopes.

Non-limiting examples of self-assembling multimeric proteins are lumazine synthase (LS) nanoparticles [Ladenstein & Morgunova, Second career of a biosynthetic enzyme: Lumazine synthase as a virus-like nanoparticle in vaccine development, Biotechnol. Rep. 27, e00494 (2020)], Ferritin nanoparticles [Wang et al., Ferritin nanoparticle-based SpyTag/SpyCatcher-enabled click vaccine for tumor immunotherapy, Nanomedicine Nanotechnol. Biol. Med. 16, 69-78 (2019); Georgiev et al., Two-Component Ferritin Nanoparticles for Multimerization of Diverse Trimeric Antigens, ACS Infect. Dis. 4, 788-796 (2018)], E2p nanoparticles [He et al., Proof of concept for rational design of hepatitis C virus E2 core nanoparticle vaccines, Sci. Adv. 6, eaaz6225 (2020)], BP26 nanoparticles [Kang et al., Antigen-Presenting, Self-Assembled Protein Nanobarrels as an Adjuvant-Free Vaccine Platform against Influenza Virus, ACS Nano 15, 10722-10732 (2021)], 0-annulus peptide nanoparticles (WO2022043449A1, [Lainšček et al., A Nanoscaffolded Spike-RBD Vaccine Provides Protection against SARS-CoV-2 with Minimal Anti-Scaffold Response, Vaccines 9, 431 (2021)]), AP205 nanoparticles [Shishovs et al., Structure of AP205 Coat Protein Reveals Circular Permutation in ssRNA Bacteriophages, J. Mol. Biol. 428, 4267-4279 (2016); Brune et al., Plug-and-Display: decoration of Virus-Like Particles via isopeptide bonds for modular immunization, Sci. Rep. 6, 19234 (2016)], encapsulin nanoparticles [Kanekiyo et al., Rational Design of an Epstein-Barr Virus Vaccine Targeting the Receptor-Binding Site, Cell 162, 1090-1100 (2015)], sHSP nanoparticle (US20070258889A1, [Flenniken et al., The Small Heat Shock Protein Cage from Methanococcus jannaschii Is a Versatile Nanoscale Platform for Genetic and Chemical Modification, Nano Lett. 3, 1573-1576 (2003)]), CCMV nanoparticles (US20070258889A1), Bacteriophage P22 nanoparticles [Patterson et al., Sortase-Mediated Ligation as a Modular Approach for the Covalent Attachment of Proteins to the Exterior of the Bacteriophage P22 Virus-like Particle, Bioconjug. Chem. 28, 2114-2124 (2017)] and synthetic nanoparticles, e.g., I3-01 [Hsia et al., Design of a hyperstable 60-subunit protein icosahedron, Nature 535, 136-139 (2016)], mutated I3-01 (mi3, CN111991556A, [Bruun et al., Engineering a Rugged Nanoscaffold To Enhance Plug-and-Display Vaccination, ACS Nano 12, 8855-8866 (2018)]), 132-19, I32-58 and I53-50 [Lacasta et al., Design and immunological evaluation of two-component protein nanoparticle vaccines for East Coast fever, Front. Immunol. 13, (2023)], or others [Lutz et al., Top-down design of protein architectures with reinforcement learning, Science 380, 266-273 (2023)].

### Conjugation Via Isopeptide Linkage

An important consideration in the design of Bm86Dock-NP is the conjugation of the Bm86Dock immunogen to the self-assembling multimeric protein such that it does not interfere with the self-assembly of the multimeric protein and lock the Bm86Dock immunogen in its pre-conjugation conformation, e.g., the 3D structure is sufficiently similar to its 3D structure in native conformation. (FIGS. 5A-5C)

In some embodiments, a Bm86Dock immunogen including a recombinant Bm86Dock module includes an amino acid sequence selected from the group consisting of SEQ ID N: 2, SEQ ID NO:3 and variants thereof conjugated via an isopeptide bond to a polypeptide subunit of a plurality of polypeptide subunits which self-assembled into a multimeric protein. In one aspect of the embodiment, the isopeptide bond is a covalently nonreversible linkage between a tag peptide and a peptide binding partner.

In some aspects of the embodiments, the tag peptide is fused to the Bm86Dock module, and the peptide binding partner is fused to a polypeptide subunit of the self-assembling multimeric protein scaffold. The tag peptide may be fused to the Bm86Dock module by a linker. Similarly, the peptide binding partner may be fused to a polypeptide subunit of the self-assembling multimeric protein scaffold by a linker.

In other aspects of the embodiments the tag peptide is fused to the polypeptide subunit of the self-assembling multimeric protein scaffold and the peptide binding partner is fused to the Bm86Dock module. The tag peptide may be fused to a polypeptide subunit of the self-assembling multimeric protein scaffold by a linker and the peptide binding partner may be fused to the Bm86Dock module by a linker.

In some aspects of the embodiments, the linker may be a flexible linker having a sequence comprising stretches of Glycine and Serine residues (GS linker). An example of a linker may have the sequence (SGG)ₙ where n is an integer ranging from 2 to 10. The length of the GS linker may be optimized to achieve appropriate separation or maintain the interaction between the peptides and the functional proteins. In a preferred embodiment, n is equal to 3 and the linker has the sequence SGGSGGSGG (SEQ ID NO: 11). The flexible linker may also contain additional amino acids such as Threonine and Alanine to maintain flexibility, as well as polar amino acids such as Lysine and Glutamic acid to improve solubility. In a preferred embodiment, the linker may have the sequence SGGGGSGSGEGSG (SEQ ID NO: 10).

Non-limiting examples of peptide tag/peptide binding partner are SpyTag/SpyCatcher, SpyCatcherΔN1, SpyCatcherΔN2, SpyCatcherΔN3, SpyCatcherΔN1C1, SpyCatcherΔN1C2, SpyTag002/SpyCatcher002, SpyTag003/SpyCatcher003, SdyTag/SdyCatcher, DogTag/DogCatcher, SnoopTag/SnoopCatcher and SilkTag/SilkCatcher.

In some embodiments, the peptide tag comprises the amino sequence having at least 90%, at least 95% or 100% sequence identity with SEQ ID NO: 4. In one aspect of the embodiment, the amino sequence of the Bm86Dock module fused to the tag peptide has an amino acid selected from the group consisting of SEQ ID NO: 5, SEQ ID NO: 6 and variants thereof.

In other embodiments, the peptide binding partner comprises the amino sequence having at least 90%, at least 95% or 100% sequence identity with SEQ ID NO: 7. In one aspect of the embodiment, the amino sequence of the peptide binding partner fused to the polypeptide subunit of the self-assembling multimeric protein scaffold has at least 90%, at least 95% or 100% sequence identity with SEQ ID NO: 8. In one aspect of the embodiment, the amino acid sequence of the polypeptide subunit of the self-assembling multimeric protein scaffold has at least 90%, at least 95% or 100% sequence identity with SEQ ID NO: 9.

TABLE 1
Sequences

| | | Description |
|---|---|---|
| SEQ ID NO: 1 | R. microplus | Immunogenic fragment |
| SEQ ID NO: 2 | R. microplus | Bm86Dock module (wild type) |
| SEQ ID NO: 3 | Synthetic Seq | Bm86Dock002 module |
| SEQ ID NO: 4 | Synthetic Seq | Tag peptide, SpyTag003 |
| SEQ ID NO: 5 | Synthetic Seq | Bm86Dock-SpyTag003 |
| SEQ ID NO: 6 | Synthetic Seq | Bm86Dock002-SpyTag003 |
| SEQ ID NO: 7 | Synthetic Seq | Peptide binding Partner, SpyCatcher003 |
| SEQ ID NO: 8 | Synthetic Seq | Recombinant LS Polypeptide subunit is |
| | | fused to SpyCatcher003 |
| SEQ ID NO: 9 | Aquifex Aeolicus | LS polypeptide subunit |
| SEQ ID NO: | Synthetic Seq | SGGGGSGSGEGSG |
| 10 | | |
| SEQ ID NO: | Synthetic Seq | SGGSGGSGG |
| 11 | | |
| SEQ ID NO: | Synthetic Seq | GSNEYYYTVS FTPNISFDSD |
| 12 | | HCKWYEDRVL EAIRTSIGKE |
| | | VFKVEILNCT Q |
| SEQ ID NO: | Synthetic Seq | DIKARLIAEK PLSKHVLRKL |
| 13 | | QACEHPIGEW CMMYPKLLIK |
| | | KNSATEIEEE N |
| SEQ ID NO: | Synthetic Seq | GSNEYYYTVS FTPNISFDSD |
| 14 | | HCKWYEDRVL EAIRTSIGKE |
| | | VFKVEILNCT QDIKARLIAK |
| | | EKPLSKHVLR |
| SEQ ID NO: | Synthetic Seq | KLQACEHPIG EWCMMYPKLL |
| 15 | | IKKNSATEIE EENSGGGGSG |
| | | SGEGSGRGVP HIVMDAYKRY K |
| SEQ ID NO: | R. microplus | LQACEHPIGE WCMMYPK |
| 16 | | |
| SEQ ID NO: | Synthetic Seq | GSNEYYYTVS FTPNISFDSD HCK |
| 17 | | |
| SEQ ID NO: | Synthetic Seq | VEILNCTQDI K |
| 18 | | |
| SEQ ID NO: | Synthetic Seq | SDHCK |
| 19 | | |

### Methods for Producing Bm86Dock-NP

In some embodiments, an expression vector may be genetically engineered to incorporate the nucleic acid sequence encoding the recombinant Bm86Dock module having the amino acid sequence selected from the group consisting of SEQ ID NO: 2, SEQ ID NO: 3, SEQ ID NO: 5, SEQ ID NO: 6 and variants thereof or encoding the subunit of the self-assembling multimeric protein comprising an amino acid sequence SEQ ID NO: 8 or SEQ ID NO: 9. Expression vectors may be selected from those readily available for use in prokaryotic or eukaryotic expression systems. Bm86Dock-NP can be produced in large-scale manufacturing via E. coli or eukaryotic systems which may include but are not limited to mammalian cells, insect cells and yeast cells.

In some embodiments, the method for producing the immunogenic Bm86Dock-NP, includes: expressing the plurality of Bm86Dock immunogens having the amino acid sequence selected from the group consisting of SEQ ID NO: 5, SEQ ID NO: 6 and variants thereof in a prokaryotic or eukaryotic expression system, independently expressing the plurality of polypeptide subunits having an amino acid sequence SEQ ID NO: 8 in a prokaryotic or eukaryotic expression system, conjugating said plurality of polypeptide subunits with said plurality of immunogens via an isopeptide bond; and allowing said plurality of polypeptide subunits conjugated to said plurality of immunogens to self-assemble into the self-assembling multimeric protein scaffold such as to display said plurality of immunogens on the outer surface of said immunogenic Bm86Dock-NP. The self-assembling multimeric protein scaffold may comprise 3, 4, 5, 6, 7, 8, 10, 12, 24, 60, 120, 180, or 240 polypeptide subunits or more.

In other embodiments, the method for producing the immunogenic Bm86Dock-NP, includes: expressing the plurality of Bm86Dock immunogens directly fused to the NP polypeptide subunits through a linker sequence as a direct protein fusion, in a prokaryotic or eukaryotic expression system; and allowing said plurality of polypeptide subunits to self-assemble into the self-assembling multimeric protein scaffold such as to display said plurality of immunogens on the outer surface of said immunogenic Bm86Dock-NP.

### Therapeutic and Prophylactic Uses

As used herein, the term “immunization” refers to the process of inducing immunity, including but not limited to producing antibodies against Bm86Dock. Immunization can be induced through vaccination. Immunization is often long-lasting and may be reactivated by repeated administration of boosters. Immunization can be used therapeutically in animals infested by ticks or prophylactically to control outbreak infestation or prevent animals from getting infested and developing tick-borne diseases.

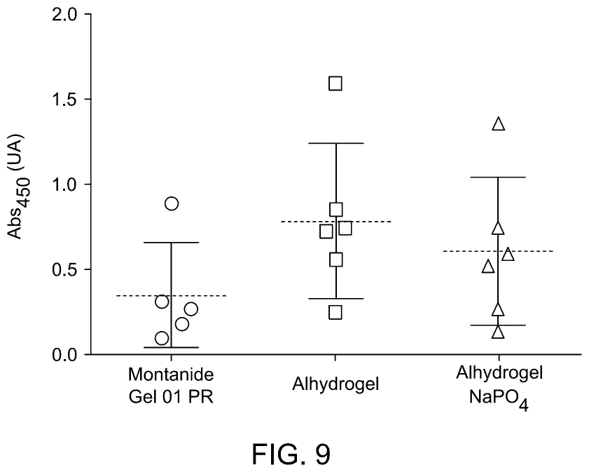

As used herein, the term “adjuvant” refers to a substance that increases the intensity of the immune response after co-administration with an immunogen. An adjuvant may act as an immunopotentiator useful for enabling immunogenic composition or vaccine to induce potent and persistent immune responses, while reducing the dose and number of boosters. Adjuvant may also increase the stability of the immunogenic composition or vaccine. In the context of the invention, an adjuvant is preferably selected from aluminum-containing adjuvants, Emulsigen range of adjuvants, liposome-based adjuvants, the Montanide Gel, Montanide ISA O/W and the Montanide range of adjuvants, Freund's complete and incomplete adjuvant, Water-in-Oil emulsions and Oil-in-Water emulsions such as MF59. In a preferred embodiment, at least one of the adjuvants is Montanide Gel 01 PR. (FIGS. 8-9). In another preferred embodiment, at least one of the adjuvants is an aluminum oxyhydroxide hydrogel suspension such as Alhydrogel®. (FIG. 9) It is understood that the type of adjuvant may be adapted to the type of animal receiving the immunogenic composition to optimize the protective immune response.

In an exemplary embodiment, the tick vaccine comprises Bm86Dock002-NP formulated with an aluminum oxyhydroxide hydrogel (AlOH) such as Alhydrogel®. (FIG. 9). In another exemplary embodiment, the tick vaccine comprises Bm86Dock-NP formulated with Montanide Gel 01 PR. (FIGS. 8-11). Such exemplary embodiments are capable of eliciting the production of protective antibodies in immunized subjects.

The present invention provides an effective design which addresses the limited effectiveness of Bm86 vaccine antigen of the prior art by using of self-assembling multimeric protein scaffold to display multiple copies of the immunogenic recombinant Bm86Dock module and as such form an immunogenic Bm86Dock-NP. The high density and structurally organized Bm86Dock immunogen array presented on the outer surface of the nanoparticle allows multiple stimulatory interactions enhancing immune protection against ticks.

In some embodiments, a Bm86Dock-NP combines a plurality of Bm86Dock modules and a self-assembling multimeric protein such that the pre-conjugation native conformational states of Bm86Dock and the self-assembling multimeric protein are preserved after conjugation and self-assembling, thereby creating a nanoparticle that is an efficacious vaccine. In some aspects of the embodiments, nanoparticles having a size ranging between 20 and 200 nm can be effectively drained to lymph nodes, facilitating a better uptake by antigen-presenting cells (APCs), thus boosting cellular responses [de Pinho Favaro et al., Recombinant vaccines in 2022: a perspective from the cell factory, Microb. Cell Factories 21, 203 (2022)].

In some embodiments, the immunogenic composition or vaccine may be administered in a subject by injection (e.g., subcutaneous, intraperitoneal or intramuscular) or orally, and may include Bm86Dock-NP as an active ingredient in an effective amount. It is understood that the appropriate effective amount of Bm86Dock-NP as well as the dosing regimen may be adjusted and optimized by a veterinarian, in the light of the relevant circumstances. A dosing regimen for prophylaxis may for example comprise administering as a prime series at least 2 doses of immunogenic composition about 3 to 4 weeks apart followed by a booster series of between 1 to 4 doses each administered 4, 6 months to 1 year apart. The first booster shot may be administered 3, 4 or 6 months after receiving the last dose of the prime series. Another dosing regimen may comprise administering as a prime series 3 doses (1×) of immunogenic composition about 30 days apart followed by a booster of 1 double dose (2×) administered 4 months after the last prime dose received.

A subject may be domestic mammals such as a mouse, a dog or a cat or livestock such as a cow, a sheep, a horse, a camel, a llama and a dromedary or a wild animal such as a capybara, a deer or a bird.

In some embodiments, the immunogenic composition or vaccine may be prepared by using pharmaceutically suitable and physiologically acceptable additives, in addition to the active ingredient, and the additives may include excipients, disintegrants, sweeteners, binders, coating agents, blowing agents, lubricants, glidants, flavoring agents, etc.

In some embodiments, the formulation of the immunogenic composition comprises Bm86Dock-NP, Tris-NaCl buffer at pH8, Montanide Gel 01 PR 15% V/V. In other embodiments, the formulation of the immunogenic composition comprises Bm86Dock-NP, Tris-NaCl buffer at pH8, Alhydrogel® 0.2% with or without NaPO₄.

In some embodiments, the vaccine or immunogenic composition may block the transmission of tick-borne pathogens from the tick to the host by interfering with specific molecular mechanisms.

In some embodiments, the immunogenic composition is used as a vaccine for the treating or preventing tick infestations and the transmission of tick-borne diseases. Tick-borne diseases are Babesiosis, Anaplasmosis, Cytauxzoonosis, Ehrlichiosis, Lyme disease, Southern Tick-Associated Rash Illness, Rickettsioses, Tick-Borne Relapsing Fever, Borrelia mayonii Disease, Typhus, Tularemia, Tick-Borne encephalitis, Powassan Virus Disease, Colorado Tick Fever, Heartland virus, Bourbon virus, Kyasanur forest disease, Omsk Hemorrhagic Fever, Crimean-Congo Hemorrhagic Fever, Severe Febrile Illness, Tick paralysis and Alpha-gal Syndrome.

### Examples

The invention is described herein by the following representative non-limiting example intended only to teach those skilled in the art the best way known to the inventors to make and use the invention. Nothing in this example or specification should be considered as limiting the scope of the present invention. The specific embodiments of the invention described may be modified or varied, without departing from the invention, as appreciated by those skilled in the art in light of the above teachings. It is therefore to be understood that, within the scope of the claims and their equivalents, the invention may be practiced otherwise than as specifically described.

### Example 1: Expression and Purification of Bm86Dock from E. coli and Mammalian Cells

Periplasmic expression of Bm86Dock in E. coli. was performed with the pelB-Bm86Dock construct and cytoplasmic expression with Trx-Bm86Dock construct. The nucleotide sequence of Bm86Dock with the peptide tag (SEQ ID NO: 5) was codon optimized and synthetized for E. coli expression. The nucleic acid was cloned between BamHI sites in vector pT7pelB [Ortega et al., Multi-Compartment and Multi-Host Vector Suite for Recombinant Protein Expression and Purification, Front. Microbiol. 9, (2018)] and pT7Trx [Correa et al., Generation of a vector suite for protein solubility screening, Front. Microbiol. 5, 67 (2014)] with T7 promoter orientation to generate pelB-Bm86Dock and Trx-Bm86Dock, respectively. FIG. 3 show SDS-PAGE 15% with the different fractions obtained during purification as follows: MW, molecular weight (with the weight indicated for relevant bands); BC, Soluble fraction (before column); FT, Flow-through; W, wash fraction from IMAC; Elu, eluted fraction form IMAC. As shown in FIG. 3A, both constructs were successfully purified from the soluble fractions by IMAC. SDS-PAGE 15% of the complete purification pipeline of Trx-Bm86Dock show: MW, molecular weight (with the weight indicated for relevant bands at the side of the gel), W, wash fraction from IMAC, Elu, eluted fraction form IMAC, E2, eluted fraction from second IMAC after TEV cleavage, C, cleaved fraction with TEV protease, FT2, Flow-through form second IMAC after TEV cleavage, BC-SE, sample before size exclusion purification, Vo, void volume, Pool-SE, pooled peaks (86.5 and 94.7 ml). The chromatogram as seen in FIG. 3B corresponds to a size exclusion chromatography on a Superdex 200 16/60 equilibrated with PBS. Arrows indicate the elution volumes for different standards being Vo, Void Volume determined with Blue dextran (>700 kDa), BSA (66 kDa) and Lysozyme (13 kDa). The eluted volumes for the different peaks are indicated on the chromatogram.

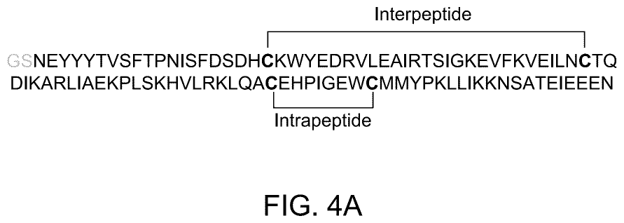

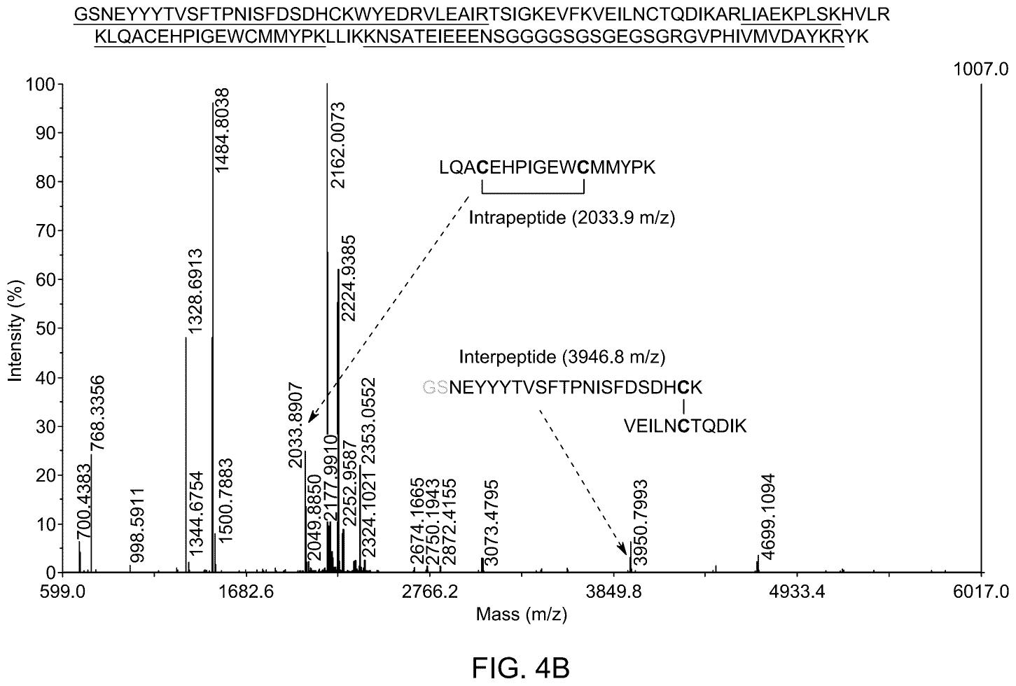

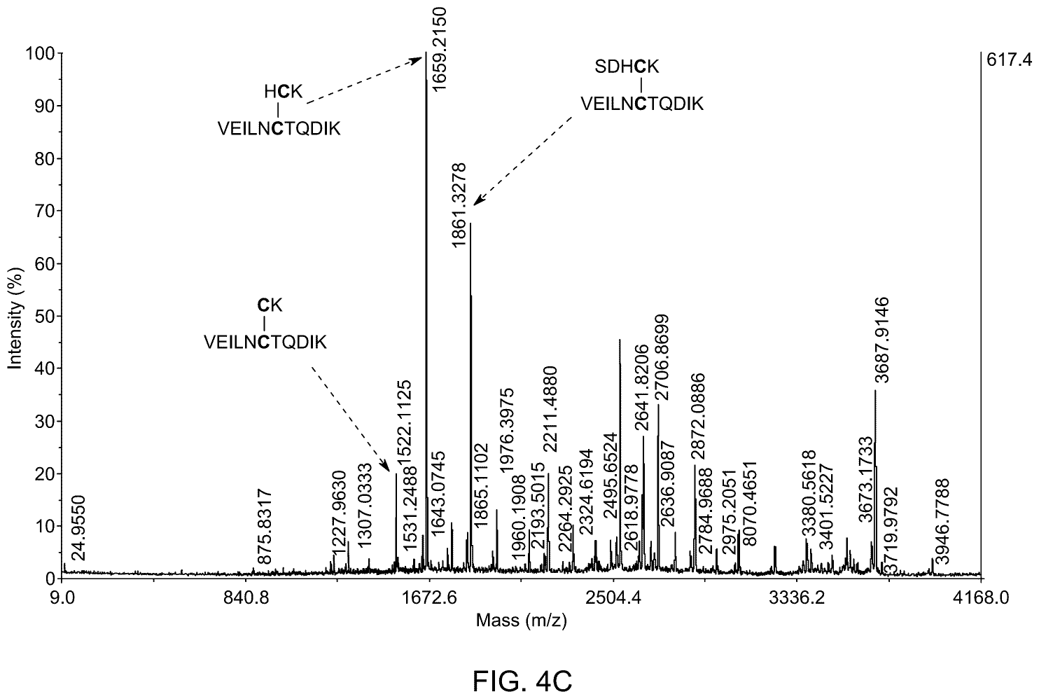

Bm86Dock was produced in both the periplasm and cytoplasm of E. coli as a soluble protein as well as a secreted protein from mammalian cells. For cytoplasmic and periplasmic expression in E. coli, the nucleic acid of the engineered polypeptide was inserted into the plasmids pT7Trx [Correa et al., Generation of a vector suite for protein solubility screening, Front. Microbiol. 5, 67 (2014)] and pT7pelB [Ortega et al., Multi-Compartment and Multi-Host Vector Suite for Recombinant Protein Expression and Purification, Front. Microbiol. 9, (2018)] and transfected into Shuffle and BL21(DE3) strains, respectively. For mammalian expression, the nucleic acid encoding for the polypeptide Bm86Dock was cloned into pCMVExFc vector [Ortega et al., Multi-Compartment and Multi-Host Vector Suite for Recombinant Protein Expression and Purification, Front. Microbiol. 9, (2018)] and transfected into HEK293 cells. In all cases the protein included a HisTag for purification by Immobilized metal affinity chromatography (IMAC). After IMAC purification, the protein was treated with Tobacco Etch Virus protease (TEV) [Rizza et al., Production, purification and characterization of a double-tagged TEV protease, Protein Expr. Purif. 191, 106021 (2022)] to cleave and remove by a second IMAC the HisTag/fusion protein from Bm86Dock. (FIG. 3A) After TEV cleavage and purification, Bm86Dock remained soluble. Moreover, the protein behaved mainly as a monomer as seen by gel filtration. (FIG. 3B) The sequence and relevant structural features, such as the two expected disulfide bonds in the predicted 3D structure (FIG. 4A), were confirmed by Mass Spectrometry. (FIGS. 4B-4C) Bm86Dock was also properly expressed and secreted from mammalian cells. (FIG. 4C) This expression system also allowed to confirm the presence of glycan post-translational modifications as theoretically suggested, which were eliminated after PNGase F treatment. This result further confirmed that the glycosylated residues were exposed as expected according to the predicted 3D structure.

### Example 2: Biophysical Characterization of Bm86Dock and Bm86Dock002

The well folded structures of the expressed Bm86Dock and Bm86Dock002 were further confirmed by circular dichroism (FIG. 7A). The spectra showed characteristic pics of secondary structure elements in folded proteins. The similarity in the spectra denoted the conservation of structure and conformational epitopes between Bm86Dock and Bm86Dock002. Upon addition of increasing concentrations of Urea as a denaturing agent, the 3D structure was lost (FIG. 7B). At a concentration of 8 M Urea, the protein was denatured, which disrupted the non-covalent interactions that hold the folded shape together, essentially causing the protein to unfold, thus resulting in the loss of any conformational epitopes. Analysis of the thermal unfolding curves showed melting temperatures of 70° C. and 84° C. for Bm86Dock and Bm86Dock002, respectively (FIG. 7C).

### Example 3: Covalent Coupling of Bm86Dock and Bm86Dock002 to a Protein Nanoparticle (NP) Via In-Vitro Ligation Methods

Bm86Dock and variants such as Bm86Dock002 were successfully expressed as single and fusion proteins which validated their predicted 3D structures. The versatility of Bm86Dock as an immunogenic module was corroborated by covalently coupling it to a nanoparticle. A tagged version of Bm86Dock (Bm86Dock-SpyTag003, [SEQ ID NO: 5]) was coupled to a N-terminal SpyCatcher003 fused Lumazine synthase scaffold (SEQ ID NO: 8). Similarly, a tagged version of variants such as Bm86Dock002 (Bm86Dock002-SpyTag003, (SEQ ID NO: 6) was coupled to a N-terminal SpyCatcher003 fused Lumazine synthase scaffold (SEQ ID NO: 8). Such design allows for the display of 60 copies of Bm86Dock or Bm86Dock002 on the outer surface of the nanoparticle. After in-vitro ligation, the functionalized nanoparticles Bm86Dock-NP and variants such as Bm86Dock002-NP were heated to 100° C. and ran in a denaturing gel, demonstrating the irreversibility of the coupling. (FIG. 6)

Relevant bands are shown on the SDS-PAGE 15% gel with the MW marker to confirm their sizes. Bm86Dock and Bm86Dock002 were incubated with NP at a 1.5:1 molar ratio for 16 hours at 4° C. (FIG. 6)

### Example 4: Enhanced Immunogenicity of Bm86Dock002 Compared to Bm86Dock after Immunization

For each immunogen Bm86Dock002 and variants such as Bm86Dock, five animals were immunized at days 0, 21 and 42 with 100 μg of protein in 2 mL volume of 12.5 mM Tris pH 8.0, 150 mM NaCl and Montanide Gel 01 PR 15% V/V. Blood samples were taken at days 0 (pre-immune) and 62. The reported difference in IgG antibody titers between days 0 and 62 for Bm86Dock was measured by ELISA. As shown in FIG. 8, the IgG titers is higher in Bm86Dock002 vaccinated animals than in Bm86Dock, suggesting that variants such as Bm86Dock002 elicit a stronger immune response than Bm86Dock.

### Example 5: Comparison in Immunogenicity of Bm86Dock-NP Vaccines Formulated with Different Adjuvants

Groups of six animals were vaccinated with Bm86Dock-NP at days 0, 21 and 42 with 200 μg of protein in 2 mL volume of 12.5 mM Tris pH 8.0, 150 mM NaCl and Montanide Gel 01 PR 15% V/V, or Alhydrogel® 0.2%, or Alhydrogel® 0.2% with 80 mM NaPO₄. Blood samples were taken at days 0 (pre-immune) and 62. The reported difference in IgG antibody titers between days 0 and 62 for Bm86Dock is measured by ELISA. As shown in FIG. 9, the IgG titers is higher in animals immunized with Bm86Dock-NP vaccine formulated with Alhydrogel® compared to vaccine formulated with Montanide Gel or Alhydrogel®/NaPO₄, suggesting an enhanced protective immune response with vaccine formulated with Alhydrogel®.

### Example 6: Bm86Dock-NP Elicits an Immune Response in Immunized Animals

The immunogenicity of both Bm86Dock and Bm86Dock-NP was tested in cattle from an experimental field at Migues-Canelones, Uruguay. Two animals per antigen were inoculated with a 100 μg dose with adjuvant Montanide Gel 01 PR, Seppic at 15% final concentration at days 0, 30 and 60. Collected serums at days 0 (pre-immune) and 90 were used for immunogenicity tests. Both Bm86Dock and Bm86Dock-NP proved to be immunogenic. (FIG. 10) Remarkably, similar antibody titers were obtained with Bm86Dock and Bm86Dock-NP, despite reducing the amount of antigen in the nanoparticle formulation by 4 times (Bm86Dock-NP). This result highlighted the benefits of the antigen functionalization.

### Example 7: Efficacy of Bm86Dock-NP

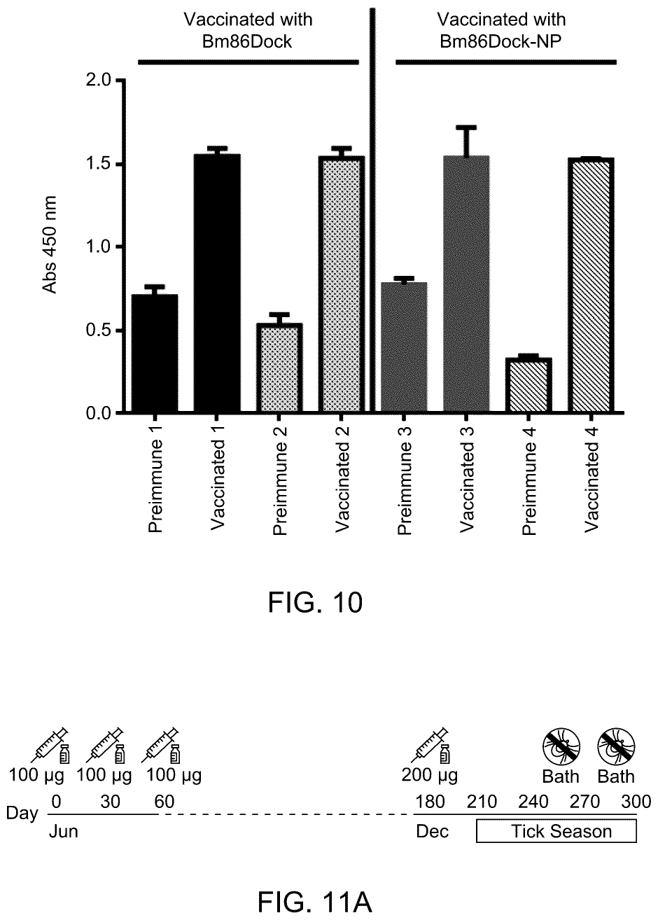

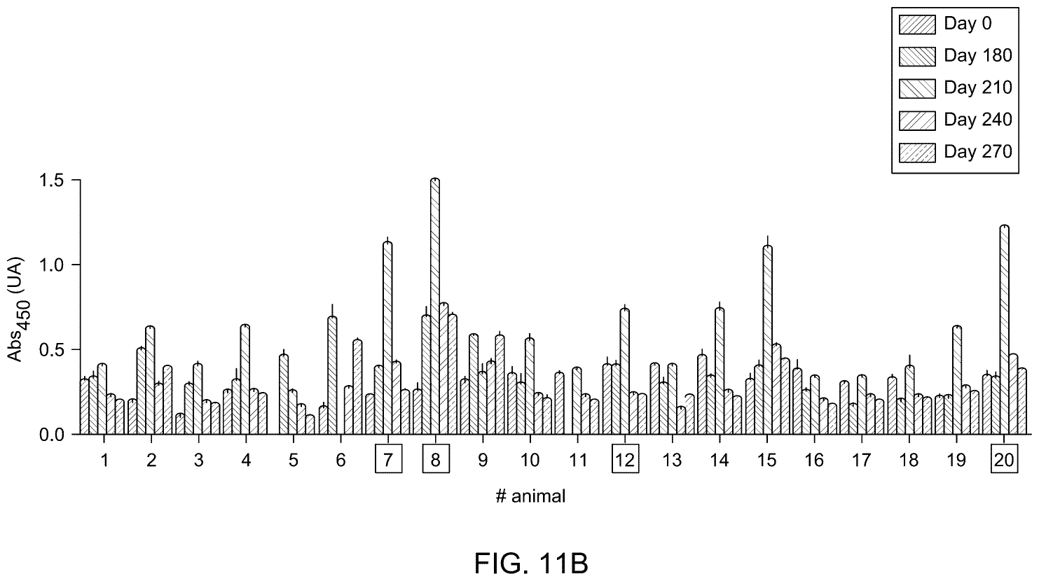

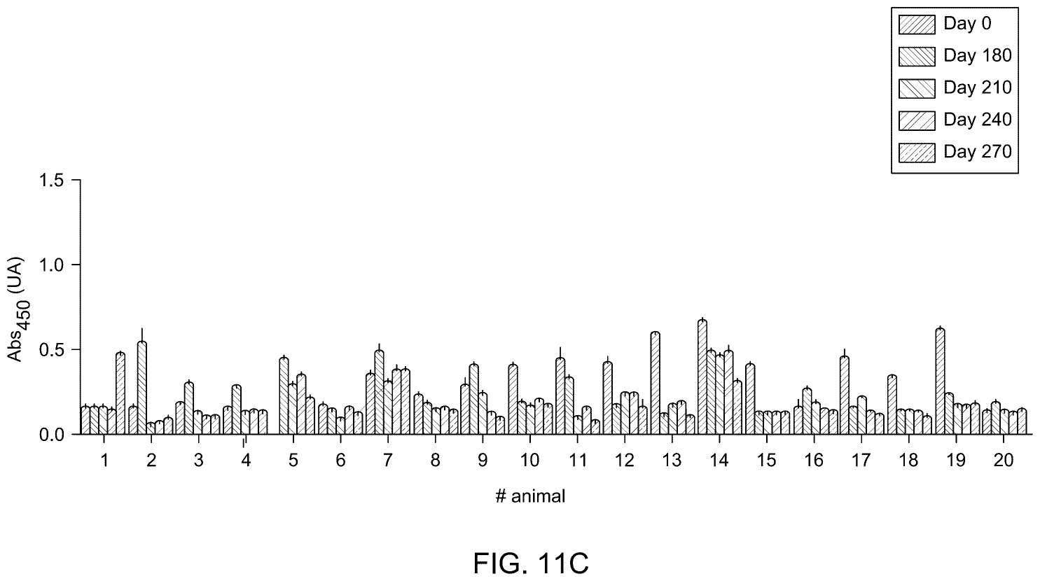

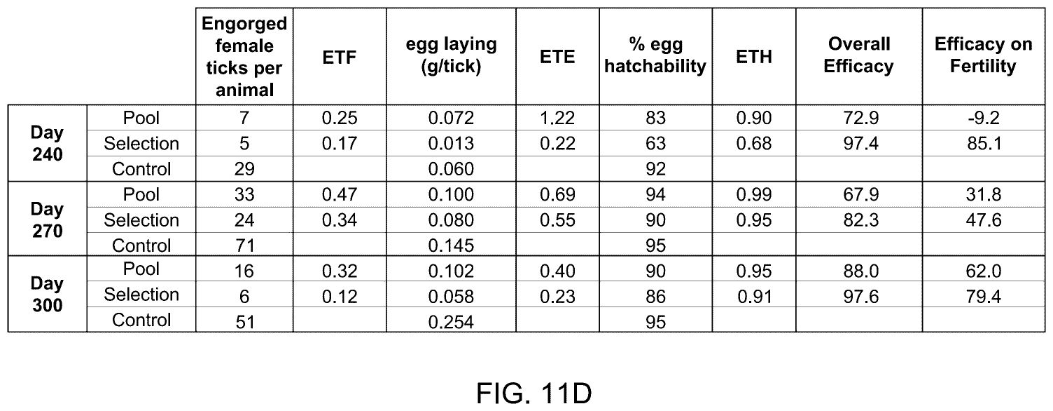

The experimental design was based on guides from [Ndawula, From Bench to Field: A Guide to Formulating and Evaluating Anti-Tick Vaccines Delving beyond Efficacy to Effectiveness, Vaccines. 9, 1185 (2021)]. The efficacy of Bm86Dock-NP was tested in a field located in Rivera, Uruguay. Two groups of 20 animals each, corresponding to 1 year old heifers, were separated, one was tested with the vaccine formulation and the other vaccinated with adjuvant only, as a control. Both groups were managed separately in two different paddocks with similar characteristics. One month before starting the trial, animals were treated with hemovaccine, to protect them against bovine sadness complex and also with acaricides to define a starting point. Three doses of 2 ml, each containing 100 μg of Bm86Dock-NP, were administered intramuscularly in the mid-neck region at 30-day intervals. At 180 days, a booster dose was administered in the same conditions as before using 200 μg of Bm86Dock-NP (FIG. 11A). Periodic blood samples were taken for each animal for serological analysis, and tick numbers were controlled for each animal (FIGS. 11B-11C). In addition, engorged female ticks were collected from animals to perform tick viability tests (egg laying and egg hatchability) (FIG. 11D). The parameters evaluated for efficacy estimation were TF (Tick feeding), TE (egg laying), TH (egg hatchability). The total efficacy was estimated by the formula: E %=100*[1−(ETF*ETE*ETH)], while efficacy over fertility is estimated by the formula: 100*[1−(ETE*ETH)]. Where ETF is defined as TF_{vaccinated}/TF_{control}, ETE is defined as: TE_{vaccinated}/TE_{control} and ETH is defined as TH_{vaccinated}/TH_{control}. The vaccine proved to have high efficacy in both a selected group of high immune responder animals (#7, #8, #12 and #20) and the pool.

The embodiments illustrated and discussed in this disclosure are intended only to teach those skilled in the art the best way known to the inventors to make and use the invention. Nothing in this disclosure should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of any claims supported by the disclosure and their equivalents, the invention may be practiced other than as specifically described.

We claim:

1. An isolated recombinant Bm86Dock module of a protein Bm86 comprising an amino acid sequence selected from the group consisting of SEQ ID NO: 2, SEQ ID NO: 3 and variants thereof, or an amino acid sequence having an immunogenic fragment consisting of SEQ ID NO: 1; wherein the recombinant Bm86Dock module elicits an immune response.

2. A Bm86Dock immunogen for treating or preventing a tick infestation in a subject comprising:
a recombinant Bm86Dock module of a protein Bm86 comprising an amino acid sequence selected from the group consisting of SEQ ID NO: 2, SEQ ID NO: 3 and variants thereof; and
a peptide tag or a peptide binding partner derived from an isopeptide protein,
wherein the recombinant Bm86Dock module elicits an immune response in the subject.

3. An immunogenic Bm86Dock-nanoparticle (Bm86Dock-NP) comprising:
a self-assembling multimeric protein scaffold composed of a plurality of polypeptide subunits, and a plurality of immunogens,
wherein each immunogen is conjugated to a polypeptide subunit of the plurality of polypeptide subunits,
wherein each immunogen of the plurality of immunogens comprises a recombinant module Bm86Dock of claim 1; and
wherein Bm86Dock-NP elicits an immune response in the subject.

4. The immunogenic Bm86Dock-NP, of claim 3, wherein the plurality of immunogens is displayed on the outer surface of the self-assembling multimeric protein scaffold, and wherein each immunogen displays at least one linear epitope and/or at least one conformational epitope of the native Bm86Dock module.

5. The immunogenic Bm86Dock-NP of claim 4, wherein the at least one linear epitope and/or the at least one conformational epitope is recognized by protective or neutralizing antibodies.

6. The immunogenetic Bm86Dock-NP of claim 3, wherein the polypeptide subunit is derived from a self-assembling multimeric protein scaffold of the group consisting of lumazine synthase (LS), ferritin, HCV envelope glycoprotein E2, Brucella outer membrane protein BP26, β-annulus peptide, AP205, encapsulin, sHSP, CCMV, bacteriophage P22, 13-01, mutated I3-01 (mi3), I32-19, I32-58 and I53-50.

7. The immunogenic Bm86Dock-NP of claim 3, wherein the self-assembling multimeric protein scaffold is composed of at least 5 polypeptide subunits, at least 24 polypeptide subunits, or at least 60 polypeptide subunits.

8. The immunogenic Bm86Dock-NP of claim 3, wherein each immunogen is conjugated to its polypeptide subunit via an isopeptide bond formed between a tag peptide and a peptide binding partner.

9. The immunogenic Bm86Dock-NP of claim 8, wherein the peptide tag/peptide binding partner is selected from the group consisting of SpyTag/SpyCatcher, SpyCatcherΔN1, SpyCatcherΔN2, SpyCatcherΔN3, SpyCatcherΔN1C1, SpyCatcherΔN1C2, SpyTag002/SpyCatcher002, SpyTag003/SpyCatcher003, SdyTag/SdyCatcher, DogTag/DogCatcher, SnoopTag/SnoopCatcher and SilkTag/SilkCatcher.

10. The immunogenic Bm86Dock-NP of claim 8, wherein the peptide tag comprises the amino sequence having at least 90%, at least 95% or 100% sequence identity with SEQ ID NO: 4.

11. The immunogenic Bm86Dock-NP of claim 8, wherein the peptide binding partner comprises the amino sequence having at least 90%, at least 95% or 100% sequence identity with SEQ ID NO: 7.

12. The immunogenic Bm86Dock-NP of claim 3, wherein each immunogen of the plurality of immunogens comprises an amino acid selected from the group consisting of SEQ ID NO: 2, SEQ ID NO: 3, SEQ ID NO: 5, SEQ ID NO: 6 and variants thereof.

13. The immunogenic Bm86Dock-NP of claim 3, wherein each polypeptide subunit of the plurality of polypeptide subunits comprises an amino acid sequence having at least 90%, at least 95% or 100% sequence identity with SEQ ID NO: 8 or SEQ ID NO: 9.

14. The Immunogenic Bm86Dock-NP of claim 3, wherein each immunogen comprising an amino acid sequence selected from the group consisting of SEQ ID NO: 2, SEQ ID NO: 3, SEQ ID NO: 5, SEQ ID NO: 6 and variants thereof is conjugated with each polypeptide subunit comprising an amino acid sequence having at least 90%, at least 95% or 100% sequence identity with SEQ ID NO: 8 or SEQ ID NO: 9 via an isopeptide bond.

15. A method for producing the immunogenic Bm86Dock-NP of claim 3, comprising: expressing the plurality of immunogens in a prokaryotic or eukaryotic expression system, independently expressing the plurality of polypeptide subunits in a prokaryotic or eukaryotic expression system, conjugating said plurality of polypeptide subunits with said plurality of immunogens via an isopeptide bond; and allowing said plurality of polypeptide subunits conjugated to said plurality of immunogens to self-assemble into the self-assembling multimeric protein scaffold such as to display said plurality of immunogens on the outer surface of said immunogenic Bm86Dock-NP,
wherein said plurality of polypeptide subunits and said plurality of immunogens are conjugated at a ratio between 1:0.5 and 1:3.

16. An immunogenic composition comprising at least one immunogenic Bm86Dock-NP of claim 3 and a pharmaceutically acceptable carrier and/or at least one adjuvant, wherein the at least one adjuvant is selected from aluminum-containing adjuvants, Emulsigen range of adjuvants, liposome-based adjuvants, the Montanide Gel, Montanide ISA O/W and the Montanide range of adjuvants, Freund's complete and incomplete adjuvant, Water-in-Oil emulsions, Oil-in-Water emulsions, and MF59.

17. The immunogenic composition of claim 16, wherein the aluminum-containing adjuvant is aluminum oxyhydroxide hydrogel (AlOH).

18. A method for preventing a tick-borne disease in a subject comprising administering an effective amount of the immunogenic composition of claim 16,
wherein the a tick-borne disease is selected from the group consisting of Babesiosis, Anaplasmosis, Cytauxzoonosis, Ehrlichiosis, Lyme disease, Southern Tick-Associated Rash Illness, Rickettsioses, Tick-Borne Relapsing Fever, Borrelia mayonii Disease, Typhus, Tularemia, Tick-Borne encephalitis, Powassan Virus Disease, Colorado Tick Fever, Heartland virus, Bourbon virus, Kyasanur forest disease, Omsk Hemorrhagic Fever, Crimean-Congo Hemorrhagic Fever, Severe Febrile Illness, Tick paralysis and Alpha-gal Syndrome.

19. A vaccine formulation comprising 5 to 200 μg of the immunogenic Bm86Dock-NP of claim 4 and aluminum oxyhydroxide hydrogel (AlOH) in storage buffer, or Tris.

20. A method for preventing tick infestations in mammals or birds that includes the intramuscular, subcutaneous or orally administrations of a vaccine comprising a Bm86Dock module having the amino acid sequence selected from the group consisting of SEQ ID NO: 2, SEQ ID NO:3 and variants thereof as a prime series of at least 3 doses approximately 3 to 4 weeks apart followed by a booster series approximately 4 months, 6 months to 1 year apart.


SEQUENCE LISTING (19 sequences)

SEQ ID NO: 1 - protein, 14 aa, Rhipicephalus microplus
LSKHVLRKLQACEH

SEQ ID NO: 2 - protein, 100 aa, Rhipicephalus microplus
NEYYYTVSFTPNISFDSDHCKWYEDRVLEAIRTSIGKEVFKVEILNCTQDIKARLIAEKPLSKHVLRKLQACEHPIGEWCMMYPKLLIKKNSATEIEEEN

SEQ ID NO: 3 - protein, 100 aa, Rhipicephalus microplus
NEYYYTVSFTPNISFDSDHCKWYEDRVLEAFRTTIGKEIFKVEILNCTQDVKARLIAEKPLSKHVLRKLQACEHPIGEWCMVYPKLLIKKNSATEIEEEN

SEQ ID NO: 4 - protein, 16 aa, synthetic construct
RGVPHIVMVDAYKRYK

SEQ ID NO: 5 - protein, 129 aa, synthetic construct
NEYYYTVSFTPNISFDSDHCKWYEDRVLEAIRTSIGKEVFKVEILNCTQDIKARLIAEKPLSKHVLRKLQACEHPIGEWCMMYPKLLIKKNSATEIEEENSGGGGSGSGEGSGRGVPHIVMVDAYKRYK

SEQ ID NO: 6 - protein, 129 aa, synthetic construct
NEYYYTVSFTPNISFDSDHCKWYEDRVLEAFRTTIGKEIFKVEILNCTQDVKARLIAEKPLSKHVLRKLQACEHPIGEWCMVYPKLLIKKNSATEIEEENSGGGGSGSGEGSGRGVPHIVMVDAYKRYK

SEQ ID NO: 7 - protein, 114 aa, synthetic construct
MVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 8 - protein, 305 aa, synthetic construct
MRGSHHHHHHGSGSENLYFQGSMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGSGGSGGSGMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 9 - protein, 154 aa, Aquifex sp.
  source 1..154: note = Aquifex aeolicus
MQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 10 - protein, 13 aa, synthetic construct
SGGGGSGSGEGSG

SEQ ID NO: 11 - protein, 9 aa, synthetic construct
SGGSGGSGG

SEQ ID NO: 12 - protein, 51 aa, synthetic construct
GSNEYYYTVSFTPNISFDSDHCKWYEDRVLEAIRTSIGKEVFKVEILNCTQ

SEQ ID NO: 13 - protein, 51 aa, synthetic construct
DIKARLIAEKPLSKHVLRKLQACEHPIGEWCMMYPKLLIKKNSATEIEEEN

SEQ ID NO: 14 - protein, 70 aa, synthetic construct
GSNEYYYTVSFTPNISFDSDHCKWYEDRVLEAIRTSIGKEVFKVEILNCTQDIKARLIAKEKPLSKHVLR

SEQ ID NO: 15 - protein, 61 aa, synthetic construct
KLQACEHPIGEWCMMYPKLLIKKNSATEIEEENSGGGGSGSGEGSGRGVPHIVMDAYKRYK

SEQ ID NO: 16 - protein, 17 aa, synthetic construct
LQACEHPIGEWCMMYPK

SEQ ID NO: 17 - protein, 23 aa, synthetic construct
  SITE 22: note = Side chain of cysteine is linked via an interpeptide linkage to cysteine of SEQ ID NO: 18.
GSNEYYYTVSFTPNISFDSDHCK

SEQ ID NO: 18 - protein, 11 aa, synthetic construct
  SITE 6: note = Side chain of cysteine is linked via an interpeptide linkage to cysteine of SEQ ID NO: 17.
  SITE 6: note = Side chain of cysteine is linked via an interpeptide linkage to cysteine of SEQ ID NO: 19.
VEILNCTQDIK

SEQ ID NO: 19 - protein, 5 aa, synthetic construct
  SITE 4: note = Side chain of cysteine is linked via an interpeptide linkage to cysteine of SEQ ID NO: 18.
SDHCK